(19) United States
(12) Patent Application Publication
(10) Pub. No.: US 2006/0014949 A1
(43) Pub. Date: Jan. 19, 2006

(54) Compositions and formulations of decitabine polymorphs and methods of use thereof

(75) Inventors: Sanjeev Redkar, Hayward, CA (US); Rajashree Joshi-Hangal, Pleasanton, CA (US)

Correspondence Address: WILSON SONSINI GOODRICH & ROSATI, 650 PAGE MILL ROAD, PALO ALTO, CA, 94304-1050, US

(73) Assignee: SuperGen Inc., Dublin, CA (US)

(21) Appl. No.: 10/890,828

(22) Filed: Jul. 13, 2004

(51) Int. Cl. C07D 251/02

(52) U.S. Cl. 544/180

(57) ABSTRACT

Pharmaceutical compositions and methods for treatment of neoplastic conditions using polymorphs of decitabine are provided. Also provided are methods for manufacturing and administering such pharmaceutical compositions.

### SUMMARY OF THE INVENTION

The present invention provides novel polymorphs of decitabine, especially crystalline anhydrate and crystalline hemihydrate forms of decitabine. The present invention also provides pharmaceutical compositions and formulations comprising such polymorphs. In some variations, the pharmaceutical compositions and formulations herein are may be adapted for administration orally, via injection and/or by inhalation. Various methods are also provided including methods of making the disclosed decitabine polymorphs, methods of manufacturing pharmaceutical formulations of the disclosed decitabine polymorphs as well as methods of using the pharmaceutical formulations for treatment of various diseases.

In one embodiment, a decitabine polymorph may be characterized by one or more of the following physical properties: X-ray powder diffraction pattern with major diffraction lines °2θ values at approximately 7.0 and 14.5 and minor diffraction lines °2θ values at approximately 13, 18.5, 21.5, 23.5 and 24.5 for Cu Kα radiation of wavelength 1.5406 Angstrom; an endotherm between about 200.5° C. and 202.5° C., an exotherm between about 202.5° C. and 204.5° C. as measured by differential scanning calorimetry; an IR spectrum with an absorption centered at about 1850 cm⁻¹ and another peak centered at about 2000 cm⁻¹; and a Raman spectra with a relatively weak stretch between about 2900 cm⁻¹ and 3000 cm⁻¹, a sharp peak at around 800 cm⁻¹, encompassed by a series of small bands from about 600 cm⁻¹ to about 1600 cm⁻¹.

In another embodiment, a decitabine polymorph may be characterized by one or more of the following physical properties: an X-ray powder diffraction pattern with major diffraction lines at °2θ values 6.5, 13.5, 17, 18, 20.5, 22.5 and 23.5 for Cu Kα radiation of wavelength 1.5406 Angstrom; an endotherm between 85° C. and 87° C., an endotherm between 93° C. and 96° C., an endotherm between 197° C. and 200° C., and an exotherm between 199° C. and 201° C. as measured by differential scanning calorimetry; an IR spectrum with a broad stretch around 3400 cm⁻¹, a stretch between 3100 cm⁻¹ and 2800 cm⁻¹, a sharp peak at around 2000 cm⁻¹ and a complex fingerprint between about 1700 cm⁻¹ and 400 cm⁻¹; and a Raman spectra with a peak between about 3100 cm⁻¹, 2800 cm⁻¹, a peak at about 800 cm⁻¹, and a series of small bands between 1600⁻¹ cm and 600 cm⁻¹.

In another embodiment, a decitabine polymorph may be characterized by one or more of the following physical properties: an X-ray powder diffraction pattern with major diffraction lines at °2θ values 13, 14.5, 16.5, 19, 23 and 27.5 for Cu Kα radiation of wavelength 1.5406 Angstrom; a first minor endotherm between 48° C. and 50° C., a second minor endotherm between 163.6° C. and 165.6° C., and a third endotherm between 194.8° C. and 196.8° C., and an exotherm between 195° C. and 197° C. as measure by differential scanning calomietry; an IR spectrum with no absorption between 3625 cm⁻¹ and 3675 cm⁻¹ a broad stretch at roughly 3400 cm⁻¹, a weak peak at 2000 cm⁻¹ and a complex fingerprint between 1700 cm⁻¹ and 500 cm⁻¹; and a Raman spectrum with a peak between about 3100 cm⁻¹ and 2800 cm⁻¹ and a peak at about 800 cm⁻¹.

### BRIEF DESCRIPTION OF THE FIGURES

FIG. 1 illustrates XRPD pattern of polymorph form A.

FIG. 2 illustrates thermal analysis of polymorph form A by differential scanning calorimetry.

FIG. 3 illustrates IR absorption spectrum of polymorph form A.

FIG. 4 illustrates Raman absorption spectrum of polymorph form A.

FIG. 5 illustrates moisture sorption/desorption data for polymorph form A.

FIG. 6 illustrates the asymmetric unit of polymorph form A.

FIG. 7 illustrates crystal packing structure of polymorph form A as viewed from the c axis.

FIG. 8 illustrates crystal packing structure of polymorph form A as viewed from the b axis.

FIG. 9 illustrates XRPD pattern of polymorph form B.

FIG. 10 illustrates thermal and differential scanning calorimetry of polymorph form B.

FIG. 11 illustrates moisture sorption/desorption data for polymorph form B.

FIG. 12 illustrates crystal packing structure of polymorph form B as viewed from the c axis.

FIG. 13 illustrates crystal packing structure of polymorph form B as viewed from the b axis.

FIG. 14 illustrates IR absorption spectrum of polymorph form B.

FIG. 15 illustrates Raman absorption spectrum of polymorph form B.

FIG. 16 illustrates XRPD pattern of polymorph form C.

FIG. 17 illustrates ¹H NMR spectroscopy of polymorph form C.

FIG. 18 illustrates thermal and differential scanning calorimetry analyses of polymorph form C.

FIG. 19 illustrates moisture sorption/desorption data for polymorph form C.

FIG. 20 illustrates a plot of the IR absorption spectrum for polymorph form C.

FIG. 21 illustrates a plot of the Raman absorption spectrum for polymorph form C

FIG. 22 illustrates a general formula of decitabine.

FIG. 23 illustrates an H NMR spectrum of a solution of decitabine polymorph form A.

FIG. 24 illustrates an H NMR spectrum of a solution of decitabine polymorph form B.

FIG. 25 illustrates an H NMR spectrum of a solution of decitabine polymorph form C.

FIG. 26 illustrates a comparison XRPD pattern of decitabine polymorph forms A (top), B (middle), and C (bottom).

FIG. 27 illustrates a comparison of IR spectrum of decitabine polymorph forms A (top), B (middle), and C (bottom).

FIG. 28 illustrates a comparison of Raman spectrum of decitabine polymorph forms A (top), B (middle), and C (bottom).

### DETAILED DESCRIPTION OF THE INVENTION

Decitabine, or 5-aza-2′-deoxycytidine, is a pyrimidine analogue that was initially synthesized in 1964. Its anti-leukemic potential was first realized by Sorm and Vesely in 1968. Recent studies have demonstrated that the anti-leukemic activity of decitabine, and its analogue 5-azacytidine, may be related to their ability to inhibit DNA methyltransferase by forming covalent adducts with the methyltransferase enzyme. The activation of silent genes is believed to be responsible for the induction of terminal differentiation of the leukemic cells leading to senescence and apoptosis. Studies show that treatment with decitabine results in phenotypic modification of the leukemic cells, a reduction of expression of CD13 and CD33 and an increase in antigenic density of surface determinants of mature myeloid cells such as CD16 and CD11c. Of interest, the expression of MHC class I molecules, HLA-DR and beta-2-microglobulin on the surface of leukemic cells is markedly increased during decitabine therapy. Therefore, decitabine treatment may increase the efficacy of an immune-mediated therapy such as IL-2 or the graft-versus-leukemia effect associated with transplantation or donor lymphocyte infusions. Decitabine is especially effective in achieving responses in patients with relapsed or refractory leukemia and is a favored drug because of its limited extramedullary toxicity.

The present invention provides novel polymorphs of decitabine. The invention further provides pharmaceutical compositions and formulations using such polymorphs. The pharmaceutical compositions and formulations are adapted for various forms of administration including oral, injection and/or inhalation. The invention also provided methods for making the novel decitabine polymorphs, methods of manufacturing pharmaceutical formulations of decitabine polymorphs and methods of treating various diseases such as, for example, leukemia and/or other conditions associated with elevated level of expression of CD13 and/or CD33 and/or reduced level of expression of CD16 and/or CD11c.

A. Definitions

To facilitate an understanding of the present invention, a number of terms and phrases are defined below:

As used herein, the term “anhydrate” refers to a compound whose empirical formula does not include water.

As used herein, the term “hemihydrate,” refers to a hydrate in which one molecule of water is associated with two molecules of decitabine.

As used herein, the term “monohydrate” refers to a compound whose empirical formula includes one water molecule.

As used herein, the term “amorphous” refers samples lacking a well-defined peak or having a broad “halo” feature in the XRPD pattern of the sample. The term “amorphous” may also refer to a material that contains too little crystal content to yield a discernable pattern by XRPD or other diffraction techniques. Glassy materials are contemplated to be amorphous. Amorphous materials do not have a true crystal lattice, and are consequently glassy rather than true solids, technically resembling very viscous non-crystalline liquids. Rather than true solids, glasses may better be described as quasi-solid amorphous material. Thus an amorphous material refers to a quasi-solid glassy material. Precipitation of a compound from solution, often effected by rapid evaporation of solvent, is known to favor amorphous forms of a compound.

As used herein, the term “broad” or “broadened” describes spectral lines including XRPD, NMR and IR spectroscopy lines is a relative term that relates to the line width of a baseline spectrum. The baseline spectrum is often that of an unmanipulated crystalline (defined below) form of a specific compound as obtained directly from a given set of physical and chemical conditions, including solvent composition and properties such as temperature and pressure, for example describing the XRPD spectrum of ground or pulverized crystalline material relative to the crystalline material prior to grinding. In materials where the constituent molecules, ions or atoms, as solvated or hydrated, are not tumbling rapidly, line broadening is indicative of increased randomness in the orientation of the chemical moieties of the compound, thus indicative of an increased amorphous content. When comparisons are made between crystalline materials obtained via different crystallization conditions, broadening indicates either increased amorphous content of the sample having the broadened spectral lines, or possibly a mixture of crystals that have similar, although not identical spectra.

As used herein, the term “crystalline” refers to a material that contains a specific compound, which may be hydrated and/or solvated, and has sufficient crystal content to exhibit a discernable diffraction pattern by XRPD or other diffraction techniques. Often, a crystalline material that is obtained from a solvent by direct crystallization of a compound dissolved in a solution or interconversion of crystals obtained under different crystallization conditions, will have crystals that contain the solvent, termed a crystalline solvate. Also, the specific solvent composition and physical properties of crystallization, collectively termed crystallization conditions, may result in crystalline material having physical and chemical properties that are unique to the crystallization conditions. Examples of crystal properties include orientation of the chemical moieties of the compound with respect to each other within the crystal and predominance of a specific form of the compound.

Depending upon the form of the specific type of crystal present, which dictates the thermodynamic stability of the crystal, various amounts of amorphous solid material containing the specific compound will be present, as a side product of the initial crystallization, and/or a product of degradation of the crystals comprising the crystalline material. Thus crystalline as used herein contemplates amorphous content of varying degrees so long as the material has a discernable diffraction pattern. Often the amorphous content of a crystalline material may be increased by grinding or pulverizing the material, which is evidenced by broadening of diffraction and other spectral lines relative to the unground crystalline material. Sufficient grinding and/or pulverizing may broaden the lines relative to the unground crystalline material to the extent that the XRPD or other crystal specific spectrum may become undiscernable, making the material substantially amorphous, or barely discernable, which may be termed quasi-amorphous.

As used herein, the term “trace” refers to an amount that is detectable by the physical and chemical detection methods employed herein, but comprises less than 0.03 of an equivalent of the specific compound present in the crystal. For example a crystalline polymorph of decitabine containing less than 0.04% (w/w) H₂O where a crystal containing one H₂O molecule per molecule of decitabine, e.g., one equivalent of H₂O, would be approximately 4.4% (w/w) H₂O is correctly described as containing a trace of water.

B. Aberrant Hypermethylation of Cancer-Related Genes

In mammalian cells, approximately 3% to 5% of the cytosine residues in genomic DNA are present in the form of 5-methylcytosine. This modification of cytosine takes place after DNA replication and is catalyzed by DNA methyltransferase using S-adenosyl-methionine as the methyl donor. Approximately 70% to 80% of 5-methylcytosine residues are found in the CpG sequence. This sequence, when found at a high frequency, in the genome, is referred to as a CpG island. Unmethylated CpG islands are associated with housekeeping genes, while the islands of many tissue-specific genes are methylated, except in the tissue where they are expressed. This methylation of DNA has been proposed to play an important role in the control of expression of different genes in eukaryotic cells during embryonic development. Consistent with this hypothesis, inhibition of DNA methylation has been found to induce differentiation in mammalian cells. Jones and Taylor, Cell, (1980) 20:85-93.

The methylated cytosine (C) in DNA, 5-methylcytosine, can undergo spontaneous deamination to form thymine (T) at a rate much higher than the deamination of cytosine to uracil. See Shen et al. Nucleic Acid Res. (1994) 22:972-976. If the deamination of 5-methylcytosine is unrepaired, it will result in a C to T transition mutation. For example, many “hot spots” of DNA damages in the human p53 gene are associated with CpG to TpG transition mutations. See Denissenko et al., Proc. Natl. Acad. Sci. USA (1997) 94:3893-1898. Other than the p53 gene, many tumor suppressor genes can also be inactivated by aberrant methylation of the CpG islands in their promoter regions. Many tumor-suppressors and other cancer-related genes have been found to be hypermethylated in human cancer cells and primary tumors. Examples of genes that participate in suppressing tumor growth and are silenced by aberrant hypermethylation include p15/INK4B (cyclin kinase inhibitor), p16/INK4A (cyclin kinase inhibitor), p73 (p53 homology), ARF/INK4A (regular level p53), Wilms tumor, von Hippel Lindau (VHL), retinoic acid receptor-β (RAR β), estrogen receptor, androgen receptor, mammary-derived growth inhibitor hypermethylated in cancer (HIC1), and retinoblastoma (Rb), Invasion/metastasis suppressor such as E-cadherin, tissue inhibitor metalloproteinase-2 (TIMP-3), mts-1 and CD44; DNA repair/detoxify carcinogens such as methylguanine methyltransferase, hMLH1 (mismatch DNA repair), glutathione S-transferase, and BRCA-1; Angiogenesis inhibitors such as thrombospondin-1 (TSP-1) and TIMP3, and tumor antigens such as MAGE-1.

In particular, silencing of p16 is frequently associated with aberrant methylation in many different types of cancers. The p16/INK4A tumor suppressor gene codes for a constitutively expressed cyclin-dependent kinase inhibitor, which plays a vital role in the control of cell cycle by the cyclin D-Rb pathway. Hamel and Hanley-Hyde, Cancer Invest. (1997) 15:143-152. P16 is located on chromosome 9p, a site that frequently undergoes loss of heterozygosity in primary lung tumors. In these cancers, it is postulated that the mechanism responsible for the inactivation of the non-deleted allele is aberrant methylation. Indeed, for lung carcinoma cell lines that did not express p16, 48% showed signs of methylation of this gene. Otterson et al. Oncogene (1995) 11:1211-1216. About 26% of primary non-small cell lung tumors showed methylation of p16. Primary tumors of the breast and colon display 31% and 40% methylation of p16, respectively. Herman et al. Cancer Res. (1995) 55:4525-4530.

Aberrant methylation of retinoic acid receptors are also attributed to development of breast cancer, lung cancer, ovarian cancer, etc. Retinoic acid receptors are nuclear transcription factors that bind to retinoic acid responsive elements (RAREs) in DNA to activate gene expression. In particular, the putative tumor suppressor RARβ gene is located at chromosome 3p24, a site that shows frequent loss of heterozygosity in breast cancer. Deng et al. (1996) Science 274:2057-2059. Transfection of RARβcDNA into some tumor cells induced terminal differentiation and reduced their tumorigenicity in nude mice. Caliaro et al., Int. J. Cancer (1994) 56:743-748; and Houle et al. Proc. Natl. Acad. Sci. USA (1993) 90:985-989. Lack of expression of the RARβgene has been reported for breast cancer and other types of cancer. Swisshelm et al., Cell Growth Differ. (1994) 5:133-141; and Crowe, Cancer Res. (1998) 58:142-148. This reason for lack of expression of RARβ gene is attributed to hypermethylation of RARβgene. Indeed, methylation of RARβ was detected in 43% of primary colon carcinomas and in 30% of primary breast carcinoma. Cote et al., Anti-Cancer Drugs (1998) 9:743-750; and Bovenzi et al., Anticancer Drugs (1999) 10:471-476.

Hypermethylation of CpG islands in the 5′-region of the estrogen receptor gene has been found in multiple tumor types. Issa et al., J. Natl. Cancer Inst. (1994) 85:1235-1240. The lack of estrogen receptor expression is a common feature of hormone unresponsive breast cancers, even in the absent of gene mutation. Roodi et al. J. Natl. Cancer Inst. (1995) 87:446-451. About 25% of primary breast tumors that were estrogen receptor-negative displayed aberrant methylation at one site within this gene. Breast carcinoma cell lines that do not express the mRNA for the estrogen receptor displayed increased levels of DNA methyltransferase and extensive methylation of the promoter region for this gene. Ottaviano et al. Cancer Res. (1994) 54:2552-2555.

Hypermethylation of human mismatch repair gene (hMLH-1) is also found in various tumors. Mismatch repair is used by the cell to increase the fidelity of DNA replication during cellular proliferation. Lack of this activity can result in mutation rates that are much higher than that observed in normal cells. Modrich and Lahue, Annu. Rev. Biochem. (1996) 65:101-133. Methylation of the promoter region of the mismatch repair gene (hMLH-1) was shown to correlate with its lack of expression in primary colon tumors, whereas normal adjacent tissue and colon tumors the expressed this gene did not show signs of its methylation. Kane et al. Cancer Res. (1997) 57:808-811.

The molecular mechanisms by which aberrant methylation of DNA takes place during tumorigenesis are not clear. It is possible that the DNA methyltransferase makes mistakes by methylating CpG islands in the nascent strand of DNA without a complementary methylated CpG in the parental strand. It is also possible that aberrant methylation may be due to the removal of CpG binding proteins that “protect” these sites from being methylated. Whatever the mechanism, the frequency of aberrant methylation is a rare event in normal mammalian cells.

C. Decitabine

Decitabine, also known as 5-aza-2′-deoxycytidine, is an antagonist of its related natural nucleoside deoxycytidine. The only structural difference between these two compounds is the presence of a nitrogen at position 5 of the cytosine ring in decitabine as compared to a carbon at this position for deoxycytidine. Two isomeric forms of decitabine can be distinguished, wherein the beta-anomer is the active form of decitabine. The modes of decomposition of decitabine in aqueous solution are (a) conversion of the active beta-anomer to the inactive α-anomer (Pompon et al. J. Chromat., (1987) 388:113-122); (b) ring cleavage of the aza-pyrimidine ring to form N-(formylamidino)-N′-beta-D-2′-deoxy-(ribofuranosy)-urea (Mojaverian and Repta, J. Pharm. Pharmacol. (1984) 36:728-733); and (c) subsequent forming of guanidine compounds (Kissinger and Stemm, J. Chromat. (1986) 353:309-318). The present application covers beta-anomers of decitabine.

Decitabine possesses multiple pharmacological characteristics. At a molecular level, it is S-phase dependent for incorporation into DNA. At a cellular level, decitabine can induce cell differentiation and exert hematological toxicity. Despite having a short half life in vivo, decitabine has excellent tissue distribution.

The most prominent function of decitabine is its ability to specifically and potently inhibit DNA methylation. As described above for methylation of cytosine in CpG islands as an example, methylation of cytosine to 5-methylcytosine occurs at the level of DNA. Inside the cell, decitabine is first converted into its active form, the phosphorylated 5-aza-deoxycytidine, by deoxycytidine kinase which is primarily synthesized during the S phase of the cell cycle. The affinity of decitabine for the catalytical site of deoxycytidine kinase is similar to the natural substrate, deoxycytidine. Momparler et al., Pharmacol. Ther. (1985) 30:287-299. After conversion to its triphosphate form by deoxycytidine kinase, decitabine is incorporated into replicating DNA at a rate similar to that of the natural substrate, dCTP. Bouchard and Momparler Mol. Pharmacol. (1983) 24:109-114.

Incorporation of decitabine into the DNA strand has a hypomethylation effect. Each class of differentiated cells has its own distinct methylation pattern. After chromosomal duplication, in order to conserve this pattern of methylation, the 5-methylcytosine on the parental strand serves to direct methylation on the complementary daughter DNA strand. Substituting the carbon at the 5 position of the cytosine for a nitrogen interferes with this normal process of DNA methylation. The replacement of 5-methylcytosine with decitabine at a specific site of methylation produces an irreversible inactivation of DNA methyltransferase, presumably due to formation of a covalent bond between the enzyme and decitabine. See Juttermann et al., Proc. Natl. Acad. Sci. USA (1994) 91:11797-11801. By specifically inhibiting DNA methyltransferase, the enzyme required for methylation, the aberrant methylation of the tumor suppressor genes can be prevented.

Incorporation of decitabine into the DNA strand has a hypomethylation effect. Each class of differentiated cells has its own distinct methylation pattern. After chromosomal duplication, in order to conserve this pattern of methylation, the 5-methylcytosine on the parental strand serves to direct methylation on the complementary daughter DNA strand. Substistuting the carbon at the 5 position of the cytosine for a nitrogen interferes with this normal process of DNA methylation. The replacement of 5-methylcytosine with decitabine at a specific site of methylation produces an irreversible inactivation of DNA methyltransferase, presumably due to formation of a covalent bond between the enzyme and decitabine. See Juttermann et al., Proc. Natl. Acad. Sci. USA (1994) 91:11797-11801. By specifically inhibiting DNA methyltransferase, the enzyme required for methylation, the aberrant methylation of the tumor suppressor genes can be prevented.

D. Decitabine Polymorphs

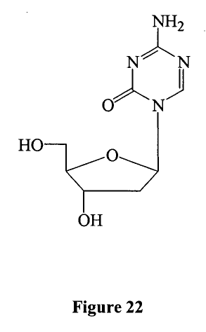

The present invention discloses various polymorphs of decitabine whose general structure is illustrated in FIG. 22. For ease, several of the polymorphs described herein are designated as polymorph forms A, B and C. In order to physically characterize these polymorphs, various tests were performed on each polymorph, including X-ray powder diffraction (“XRPD”), variable-temperature X-ray powder diffraction (“VT-XRPD”), thermal analysis (“TA”), differential scanning calorimetry (“DSC”), infrared spectrometry (“IR”), Raman spectrometry (“Raman”), NMR spectroscopy, moisture sorption/desorption analysis (“MS/DA”) and hot stage microscopy.

The decitabine polymorphs of the present invention may be obtained by direct crystallization of decitabine or by crystallization followed by interconversion. In particular, a solution was prepared by almost dissolving 35.5 mg of SSCI-15003 (decitabine obtained from from SuperGen Inc. (Lot. No. H113210/27262A). The solution was filtered into a vial, which was then sealed and allowed to cool to ambient temperature. Solids are formed overnight.

In some instances, the polymorphs that result are crystalline anhydrate, monohydrate and hemihydrates. Amorphous polymorphs may also be derived by rapidly evaporating solvent from solvated decitabine, or by grinding, pulverizing or otherwise physically pressurizing or abrading any of the various crystalline polymorphs described herein. General organic methods for precipitating and crystallizing organic compounds may be applied to preparing the various decitabine polymorphs. These general methods are known to those skilled in the art of synthetic organic chemistry and pharmaceutical formulation, and are described, for example, by J. March, “Advanced Organic Chemistry: Reactions, Mechanisms and Structure,” 4th Ed. (New York: Wiley-Interscience, 1992).

1. Polymorph Form A of Decitabine

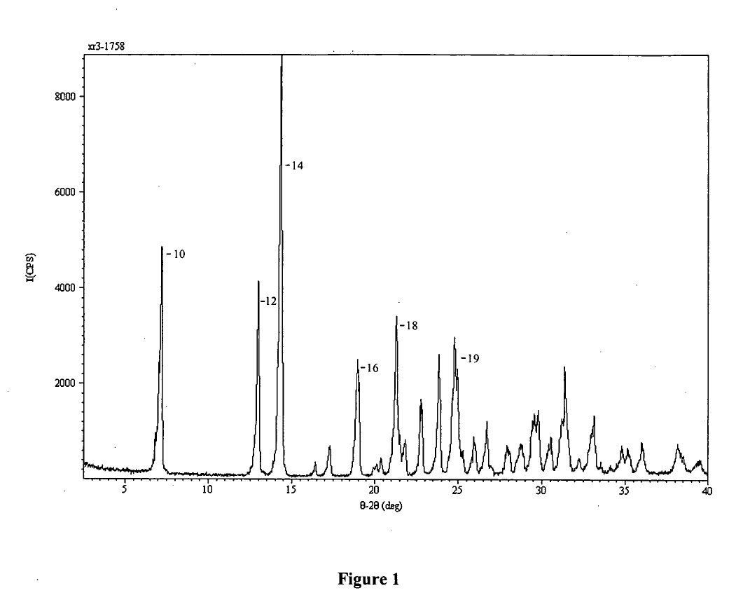

Decitabine polymorph form A can be obtained from SuperGen Inc. (Lot. No. H113210/27262A). Form A is a crystalline anhydrate as is evident by the presence of peaks in the XRPD pattern for the sample. FIG. 1 illustrates the XRPD pattern of Form A. Major diffraction lines 10 and 14 are observed at approximately 7 and 14.5 °2θ, respectively. Sharp, but weaker lines 12, 16, 18 and 19 are observed at 13, 18.5, 21.5 and 24.5 °2θ, respectively. Form A exhibits needle morphology between °2θ values of 25 and 40. Furthermore, consistent with this data, form A exhibits a preferred orientation effect observed as variations in relative peak intensity which is often observed in crystalline materials having a needle or plate morphology.

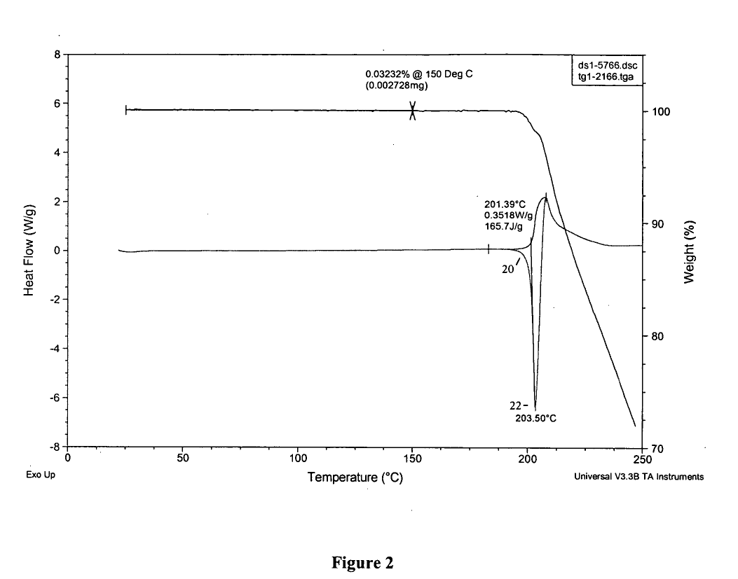

Thermal analysis of form A further suggests that this polymorph is an anhydrate of decitabine. Thermal analysis and DSC results are summarized in Table 1 below and in FIG. 2.

TABLE 1
Thermal Data on Crystal Form A

| Form | DSC Results* | TA Results** |
|---|---|---|
| A | Endo 203.4, exo 201.4 | <0.1% |

*endo—endotherm, exo—exotherm, maximum temperature reported for transition
**percent weight change from 25 to 150° C.

Thermal analysis of form A does not show a weight loss up to the decomposing point of the sample at roughly 200° C. Thus in some embodiments, polymorphic form A of decitabine is further characterizable by differential scanning calorimetry, as having an endotherm at between 198° C. and 208° C. at a rate of 10° C./min. More preferably, polymorphic form A of decitabine is characterizable by differential scanning calorimetry, as having an endotherm at between 200° C. and 205° C. at a rate of 10° C./min. Or more preferably, polymorphic form A of decitabine is characterizable by differential scanning calorimetry, as having an endotherm at between 202° C. and 204° C. at a rate of 10° C./min.

The above endotherm is accompanied by an exothermic event, which is around 199° C. to 206° C., or more preferably 201° C. to 204° C., or more preferably around 203.5° C. This behavior indicates that form A begins to melt with decomposition or crystal reordering at about 197° C. to about 199° C. or more preferably at about 198.2° C., and has a melting point of about 199° C. to about 201° C., or more preferably about 200° C. A melting point near 200° C. is also confirmed by hot stage data, summarized below in Table 2.

TABLE 2
Hot Stage Microscopy Observations

| Form | Sample # | Observations |
|---|---|---|
| A | 1 | Needles darken between 185° C. and 198° C., |
| | | melt onset 198.2° C., melt at 200.1° C. |

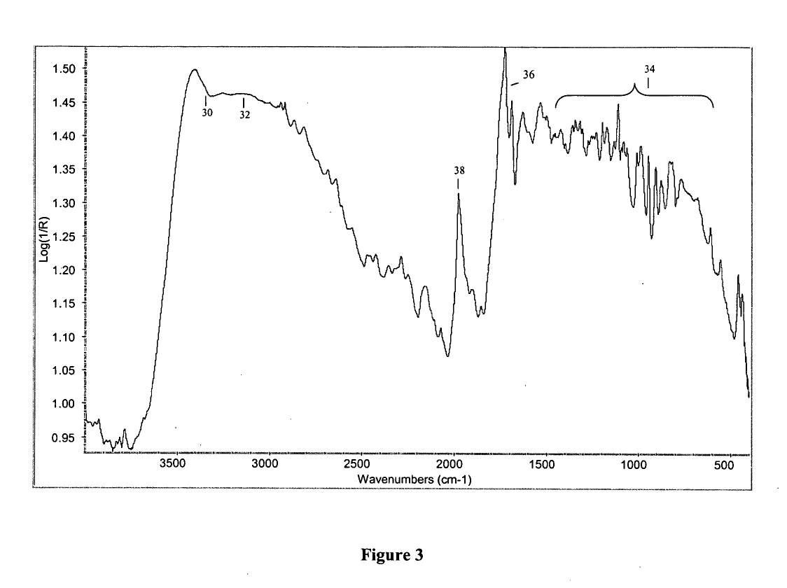

The IR spectrum for Form A is plotted in FIG. 3. The spectrum shows a peak 30 around 3500 cm⁻¹, and a broad stretch 32 between 3500 and 3000 cm⁻¹, a complex fingerprint region 34 between 1700 cm⁻¹ and 400 cm⁻¹, and minimal absorption between about 3700 cm⁻¹ and 4000 cm⁻¹. Furthermore, the IR spectrum illustrates a distinctive peak 36 at approximately 1850 cm⁻¹ and 38 at approximately 2000 cm⁻¹.

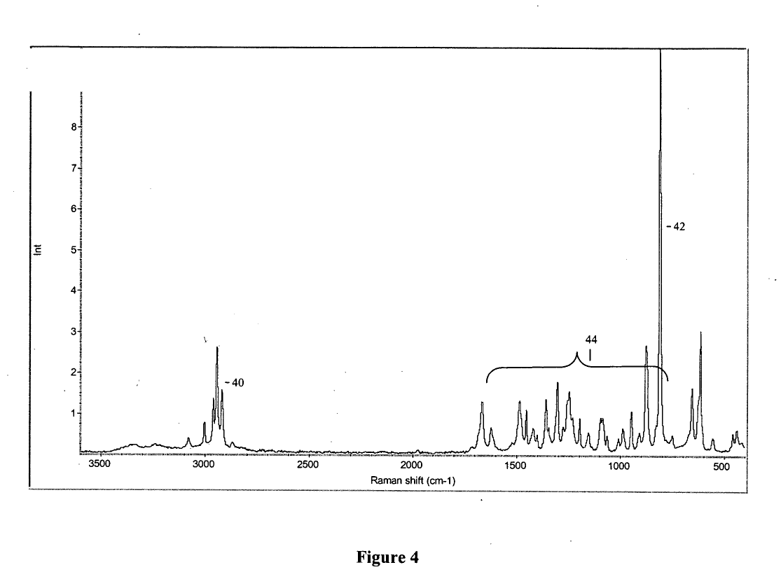

The Raman spectrum for Form A is provided in FIG. 4. The Raman spectrum shows relatively weak stretch 40 between 3000 cm⁻¹ and 2900 cm⁻¹, a sharp peak 42 at approximately 800 cm⁻¹, and a series of smaller bands 44 in the region from 600 cm⁻¹ to 1.600 cm⁻¹.

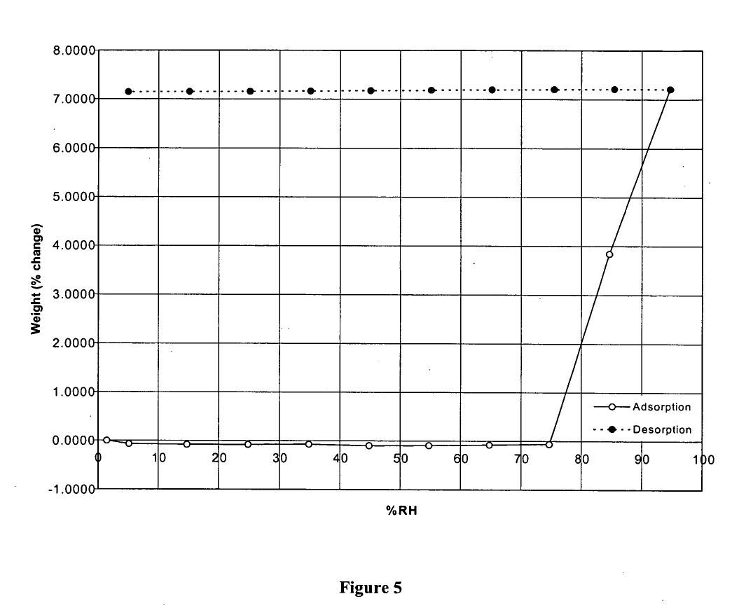

Moisture sorption/desorption analysis of form A demonstrates that this solid phase polymorph is unstable relative to its hydration to decitabine monohydrate (form B). Data of moisture sorption/desorption for Form A are summarized below in Table 3 and also in FIG. 5.

TABLE 3
Moisture Sorption/Desorption Data of Polymorph Form A.

| Elap Time | Weight | Weight | Samp Temp | Samp |
|---|---|---|---|---|
| Min | Mg | % chg | Deg C. | RH % |
| 0.0 | 8.9301 | 0.0000 | 24.92 | 1.46 |
| 27.9 | 8.9239 | −0.0698 | 24.92 | 5.14 |
| 41.6 | 8.9231 | −0.0784 | 24.92 | 14.81 |
| 51.2 | 8.9229 | −0.0804 | 24.92 | 24.88 |
| 62.2 | 8.9236 | −0.0733 | 24.92 | 34.89 |
| 82.2 | 8.9212 | −0.1004 | 24.91 | 44.86 |
| 93.2 | 8.9218 | −0.0931 | 24.91 | 54.83 |
| 107.7 | 8.9232 | −0.0775 | 24.91 | 64.82 |
| 118.3 | 8.9244 | −0.0646 | 24.91 | 74.73 |
| 304.2 | 9.2729 | 3.8389 | 24.91 | 84.63 |
| 433.1 | 9.5740 | 7.2104 | 24.90 | 94.67 |
| 443.8 | 9.5740 | 7.2106 | 24.89 | 85.43 |
| 454.8 | 9.5733 | 7.2022 | 24.88 | 75.49 |
| 467.8 | 9.5724 | 7.1925 | 24.88 | 65.19 |
| 480.8 | 9.5716 | 7.1837 | 24.88 | 55.14 |
| 493.8 | 9.5709 | 7.1753 | 24.87 | 45.07 |
| 504.8 | 9.5702 | 7.1671 | 24.88 | 35.12 |
| 515.4 | 9.5694 | 7.1586 | 24.88 | 25.13 |
| 524.9 | 9.5689 | 7.1528 | 24.89 | 15.15 |
| 533.4 | 9.5684 | 7.1472 | 24.89 | 4.93 |

As Table 3 illustrates form A loses a minimal amount of water (0.06%) upon equilibration to 5% RH. The material loses a total of about 0.1% in the region from 5 to 45% RH. Furthermore, as is illustrated in FIG. 5, a sample of form A beings moisture sorption above 75% RH with a total weight gain of 7.3% from 5 to 95% RH. Essentially all of the mass gained in the sorption event may be retained during the desorption cycle as decitabine polymorph form B. While experiments preformed on form A indicate that atmospheric moisture is capable of partially hydrating form A to form B, compression of form A at about 10,000 psi for approximately an hour did not induce a form change. Thus form A can be physically stable during tableting.

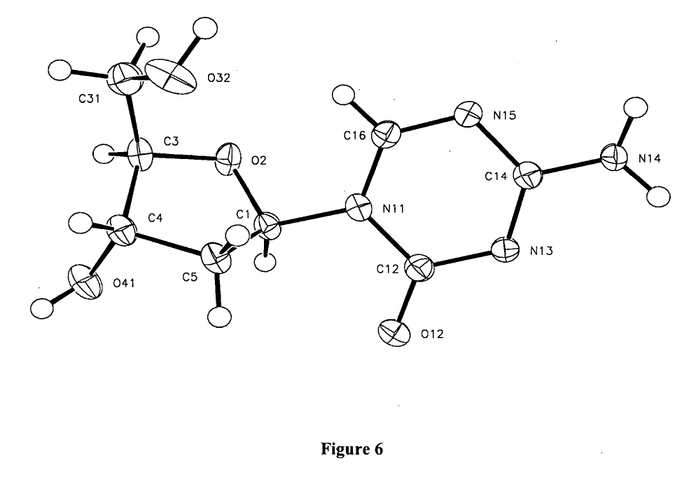

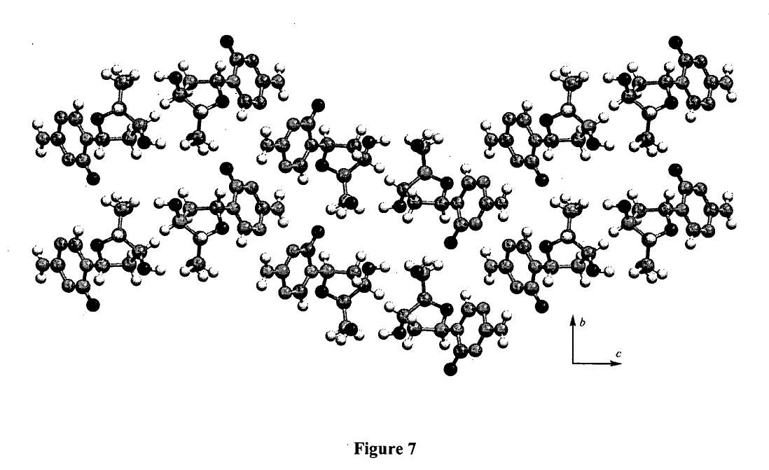

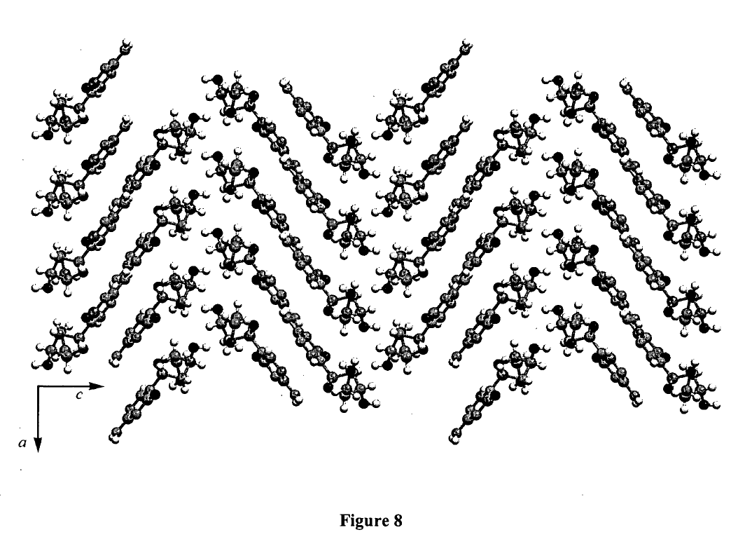

A single crystal of form A was grown by cooling a solution of decitabine in methanol. Crystal X-ray structure for the solid form was obtained. The asymmetric unit of form A is illustrated in FIG. 6 Furthermore, polymorph form A of decitabine is characterized by a crystal packing structure of corrugated tape that results from hydrogen bonding between the azocytosine rings. The packing structure of form A of decitabine as viewed down the c axis is illustrated in FIG. 7. The packing structure of form A of decitabine as viewed down the b axis is illustrated in FIG. 8.

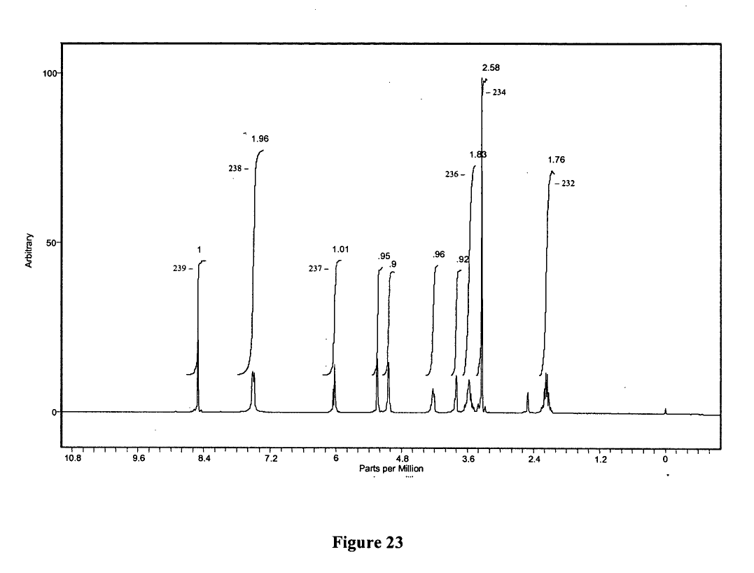

Solution state H ¹NMR spectra was obtained at ambient temperature. Form A H ¹NMR results are illustrated in FIG. 23 with shifts expressed in parts per million. Shifts 238 and 239 indicate electronic properties of an aromatic ring. Shift 237 indicates hydrogens directly attached to C═C double bonds. Shifts 234 and 236 indicate an ether region or also alcohols and esters. Finally, shift 232 indicates a carbonyl region having protons attached to carbons next to a C═O, C═C or phenyl ring. If there are more than one electronegative substituents, the proton may come even further downfield. Each of these groups induces a slight polarization of the C—H bond, decreasing electron density and deshielding the proton.

2. Polymorph Form B of Decitabine

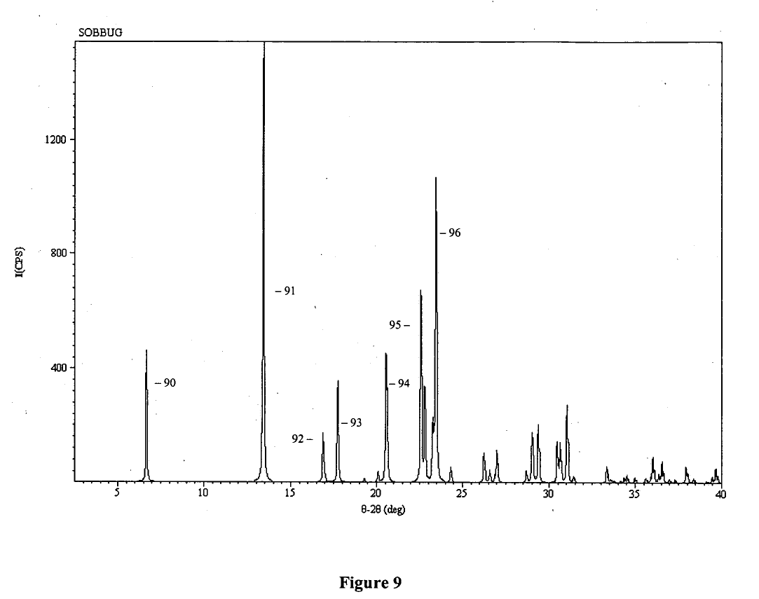

As discussed above, decitabine is able to crystallize as a monohydrate, which is designated form B. Form B polymorph of decitabine may be prepared by exposing form A to high relative humidity followed by crystallization of the monohydrate form. In one example, form A converts into form B in aqueous salt solution of sodium chloride at 75.5% RH, at 20° C. Form B is thought to be a monohydrate and is illustrated in FIG. 9. The XRPD pattern of form B has diffraction lines 90-96 at about 6.5, 13.5, 17, 18, 20.5, 22.5, and 23.5 values of °2θ, respectively.

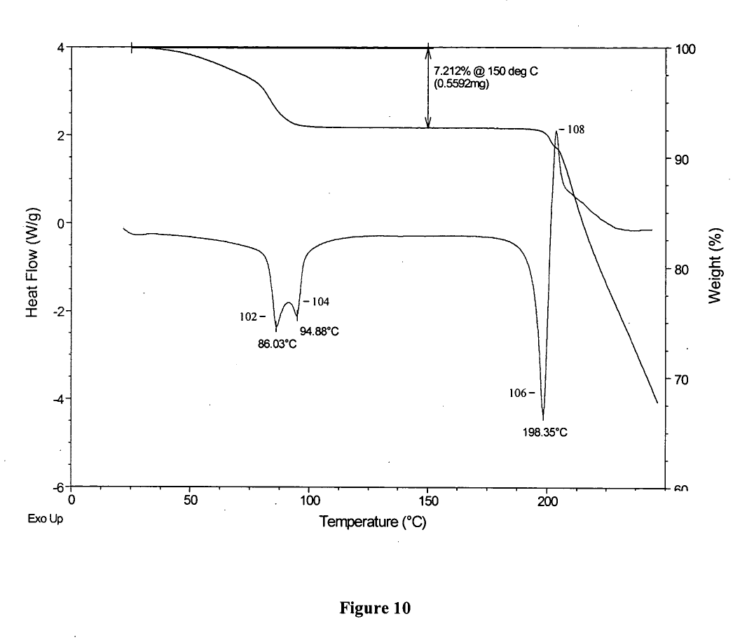

Thermal analysis and DSC data on Form B are provided below in Table 4 and plotted in FIG. 10.

TABLE 4
Thermal Data on Crystal Form B

| Form | DSC Results* | TGA Results** |
|---|---|---|
| B | endo 86.0, 94.9, 198.4 | 7.212% |
| | exo 200 | |

*endo—endotherm, exo—exotherm, maximum temperature reported for transition
**percent weight change from 25 to 150° C.

The thermal analysis data of polymorph form B indicates that the crystalline water in the sample is removed at temperature below about 100° C. The calculated weight loss at 150° C. of 7.2% is in agreement with the theoretical weight change of 7.3% associated with the desolvation of a monohydrate to an anhydrate. The DSC curve for Form B illustrated in FIG. 10 shows two endothermic events 102 and 104 at 86° C. and 94.9° C., respectively. The endothermic event associated with melting/decomposition of form B is slightly lower than the endothermic event observed in the DSC plot for form A. These endothermic events of form B are assigned to loss of water and are followed by a sharp endotherm 106 at 198.35° C. and an exotherm 108 at 200° C., assigned to a possible melt/recrystallization. A sample of form B heated to a temperature of approximately 150° C. for ten minutes and then allowed to cool to room temperature converts to form A. This demonstrates that form A can be produced from form B if desired. On the other hand, form B converts to form C upon storage in a vacuum oven at room temperature for 6 days, and VT-XRPD experiments demonstrate that form B will partially heat to generate polymorph form C.

Thus, in some embodiments, the decitabine polymorph B is characterizable by a differential scanning calorimetry having an endotherm between 81° C. and 91° C., an endotherm between 90° C. and 100° C., an endotherm between 193° C. and 203° C. More preferably, the decitabine polymorph B is characterizable by a differential scanning calorimetry having an endotherm between 83° C. and 88° C., an endotherm between 93° C. and 98° C., and an endotherm between 195° C. and 200° C. Or more preferably, the decitabine polymorph B is characterizable by a differential scanning calorimetry as having an endotherm between 85° C. and 87° C., an endotherm between 94° C. and 96° C., and an endotherm between 197.4° C. and 199.4 C.

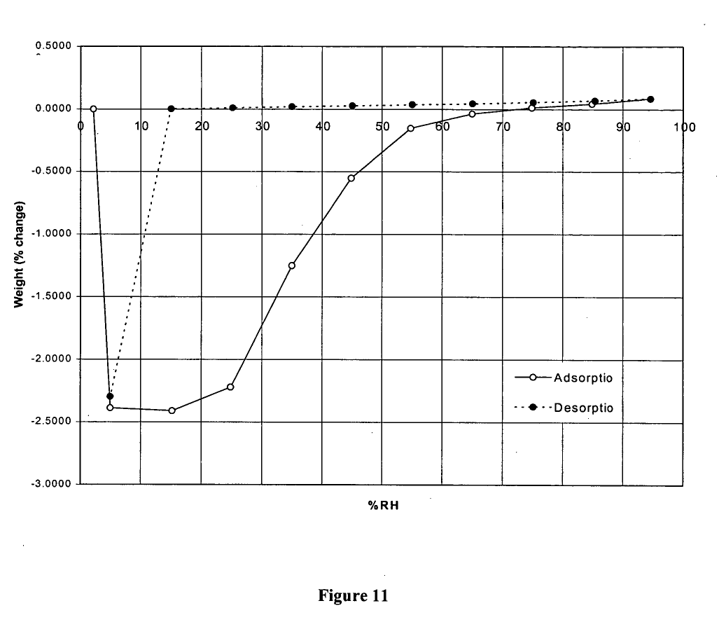

Moisture sorption/desorption data for Form B is provided below in Table 5 below and also in FIG. 11.

TABLE 5
Moisture Sorption/Desorption Data for Form B

| Elap Time | Weight | Weight | Samp Temp | Samp |
|---|---|---|---|---|
| Min | Mg | % chg | Deg C. | RH % |
| 0.0 | 9.6711 | 0.0000 | 25.08 | 2.15 |
| 185.1 | 9.4400 | −2.3894 | 25.06 | 4.98 |
| 193.2 | 9.4378 | −2.4118 | 25.07 | 15.24 |
| 242.2 | 9.4561 | −2.2223 | 25.07 | 24.84 |
| 384.9 | 9.5500 | −1.2517 | 25.06 | 35.00 |
| 499.6 | 9.6177 | −0.5520 | 25.06 | 44.84 |
| 556.5 | 9.6562 | −0.1535 | 25.07 | 54.81 |
| 579.8 | 9.6675 | −0.0371 | 25.07 | 65.01 |
| 592.3 | 9.6722 | 0.0119 | 25.08 | 74.89 |
| 601.5 | 9.6752 | 0.0426 | 25.08 | 84.81 |
| 613.0 | 9.6793 | 0.0851 | 25.09 | 94.59 |
| 619.7 | 9.6777 | 0.0688 | 25.07 | 85.33 |
| 627.7 | 9.6764 | 0.0553 | 25.06 | 75.10 |
| 635.7 | 9.6753 | 0.0439 | 25.07 | 65.04 |
| 643.2 | 9.6745 | 0.0357 | 25.06 | 55.06 |
| 650.7 | 9.6737 | 0.0274 | 25.06 | 45.01 |
| 658.2 | 9.6728 | 0.0184 | 25.07 | 35.00 |
| 665.2 | 9.6720 | 0.0098 | 25.06 | 25.12 |
| 672.3 | 9.6711 | 0.0005 | 25.07 | 15.05 |
| 854.2 | 9.4488 | −2.2984 | 25.07 | 4.98 |

This data indicates that form B may partially desolvate at 5% RH. Form B loses about (2.4%) of water upon equilibration to 5% RH, but regains that moisture at about 44% RH and further regains (0.09%) of water at 95% RH. While form B was stable at 5% RH, it underwent a partial form change to provide a mixture of forms B and form C. Based on the characterization data, form B is a monohydrate of form A.

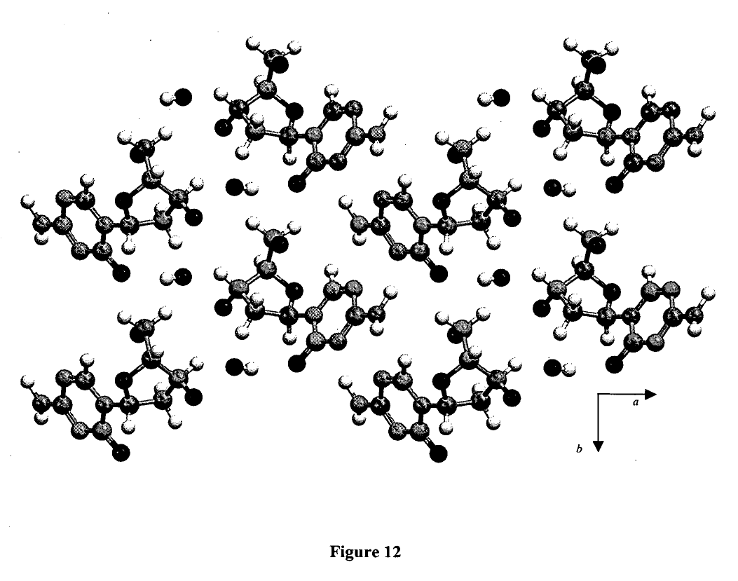

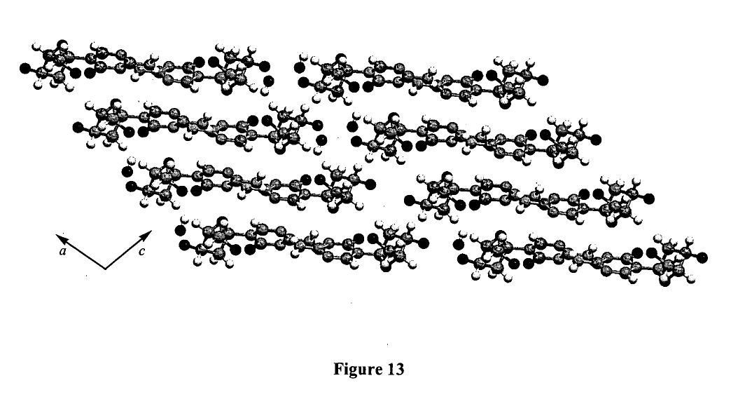

Single-crystal X-ray data for decitabine polymorph form B was used to generate packing diagrams illustrated in FIGS. 12-13. FIG. 12 illustrates the packing diagram of decitabine form B when viewed down the c axis. FIG. 13 illustrates the packing diagram of decitabine polymorph form B when viewed down the b axis. The dominant interaction in form B are the hydrogen bonds that defined the one-dominational corrugated tape structure that is also found in form A. However, form B has longer (e.g., weaker) hydrogen bonds between azacytosine rings than those of form A. Moreover, unlike what is observed in the structure of form A, the deoxyribose rings in form B are hydrogen bonded to water molecules that separate adjacent tape units. The corrugated motif that is observed for form A is also not present in form B. Instead, the tape units for form B are stacked along the same plane. If the water molecules in form B are removed from the structure, the compound must undergo significant additional molecular rearrangements in order to convert to form A.

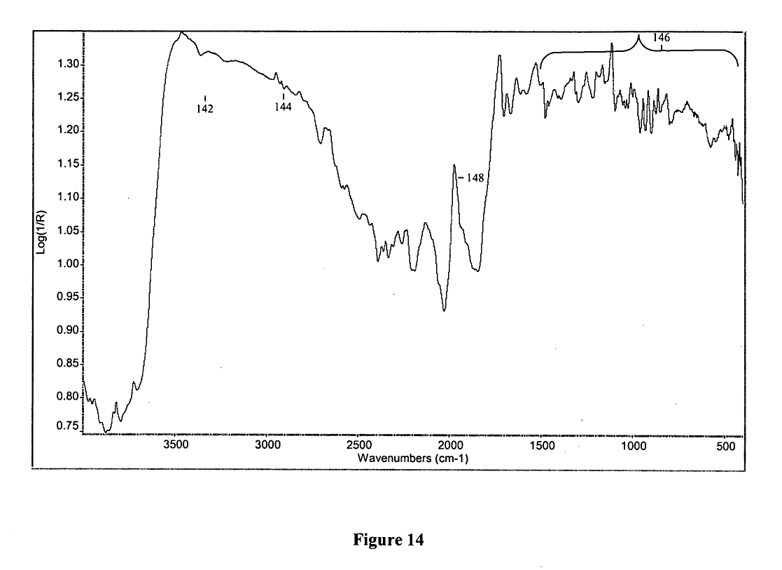

FIG. 14 illustrates an IR spectrum for form B. The IR spectrum demonstrates a relatively broad OH stretch 142 around 3400 cm⁻¹. The aromatic and aliphatic CH stretches 144 between 3100 and 2800 cm⁻¹ are also broad. The spectrum has a complex fingerprint region 146 and 1700 cm⁻¹ and 400 cm⁻¹. A sharp peak 148 at approximately 2000 cm⁻¹, represents a C═C stretch (such as in aliphatic ring).

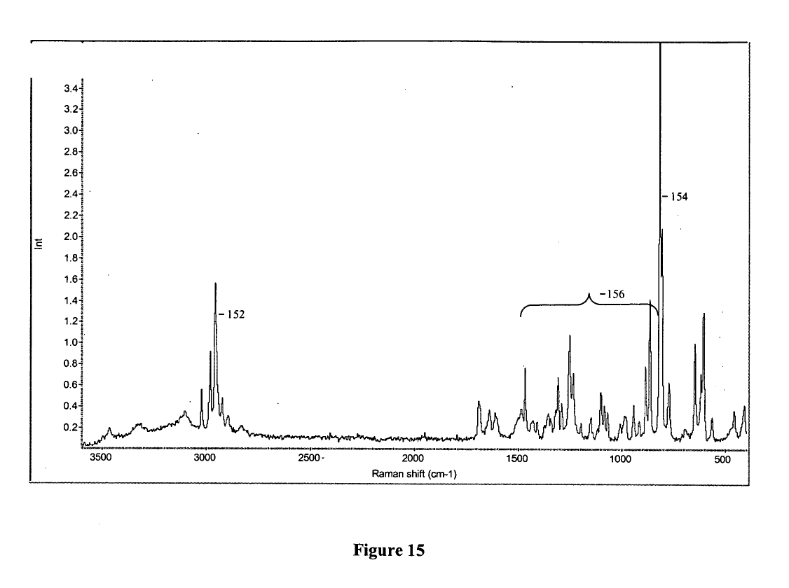

The Raman spectrum of form B is provided in FIG. 15. The Raman spectrum shows relatively weak aromatic and aliphatic CH stretches 152 between 3100 and 2800 cm⁻¹, a peak 154 at about 800 cm⁻¹ indicating a C—O—C bond, and a series of small bands between 1600 cm⁻¹ and 600 cm⁻¹ illustrating aliphatic and alicyclic chain vibrations.

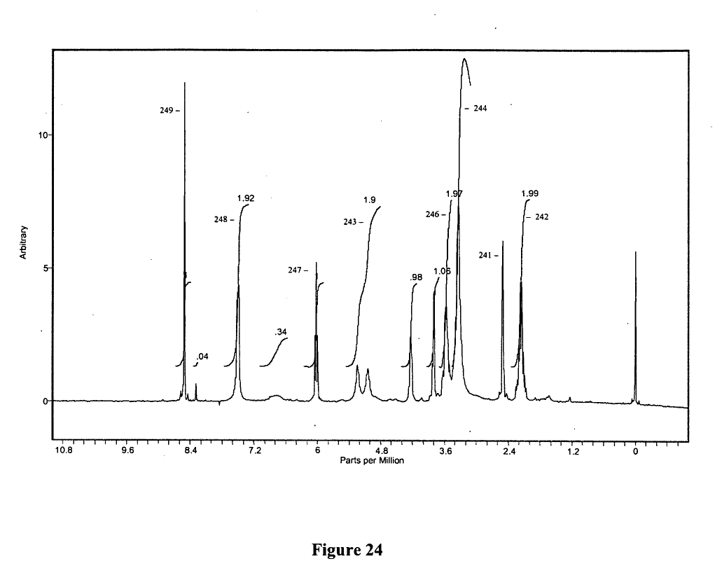

Furthermore, ¹H NMR analysis of a solution of form B dissolved in methyl sulfoxide-d₆ confirms that the sample of form B prepared in this manner is chemically pure. See FIG. 24. FIG. 24 illustrates ¹H NMR shifts of decitabine polymorph form B. Shifts 248 and 249 indicate electronic properties of an aromatic ring with shift 248 having a higher peak than that of polymorph form B. Shift 247 indicates hydrogens directly attached to C═C double bonds. Shifts 244 and 246 indicate an ether region or also alcohols and esters. Finally, shift 242 and 241 indicates a carbonyl region having protons attached to carbons next to a C═O, C═C or phenyl ring.

3. Polymorph Form C of Decitabine

Polymorph form C can be obtained from Supergen Inc. (Lot No. 97045sg04) or may be produced from decitabine polymorph form B as described above.

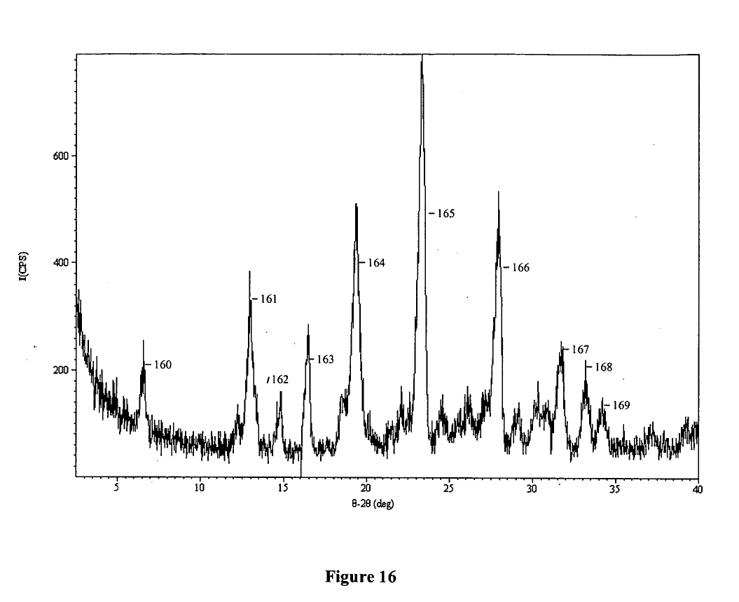

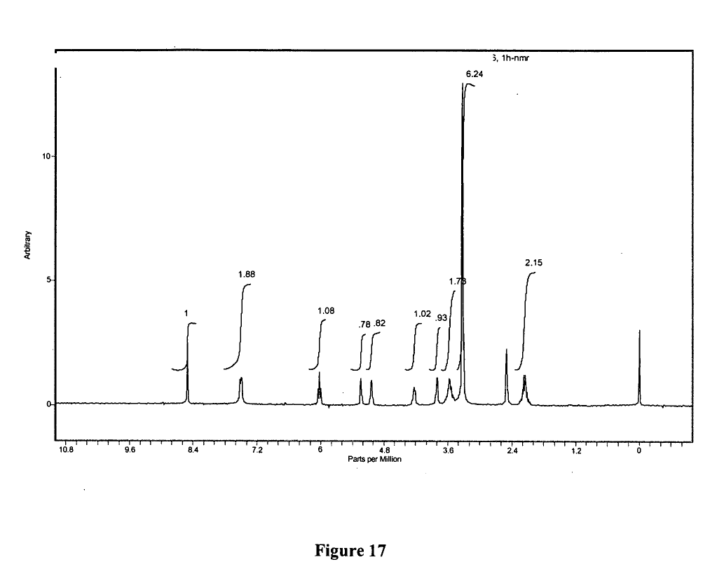

The XRPD pattern of Form C is provided in FIG. 16. The form C polymorph pattern has major diffraction lines 160-169 at about 6, 13, 14.5, 16.5, 19, 23, 27.5, 32, 33, and 34 values of °2θ, respectively. This pattern was found to be chemically pure upon analysis by solution ¹H NMR spectroscopy as is provided in FIG. 17.

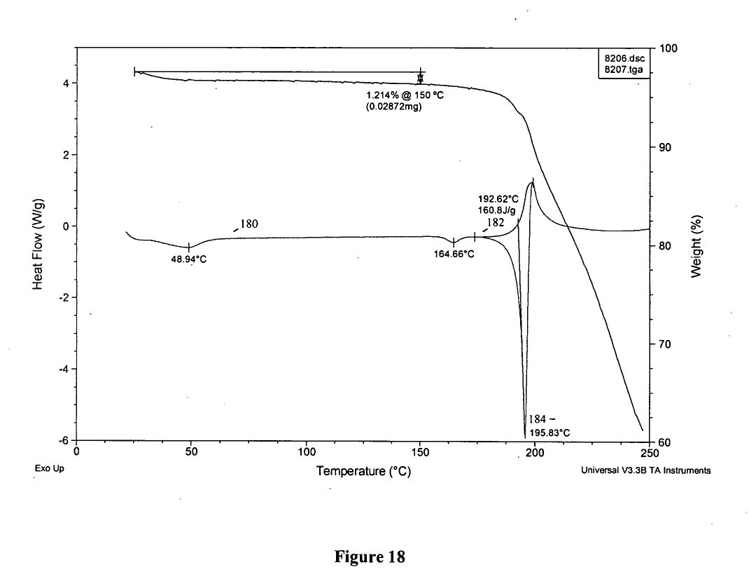

The TGA data for form C is provided below in Table 6 and in FIG. 18.

TABLE 6
Thermal Data on Crystal Form C

| Form | DSC Results* | TGA Results** |
|---|---|---|
| C | Endo 49.3° C., 164.6° C., | 1.2% |
| | and 195.8° C. | |
| | Exo 196° C. | |

*endo—endotherm, exo—exotherm, maximum temperature reported for transition
**percent weight change from 25 to 150° C.

Weak endotherms 180 and 182 are observed between 48° C. and 50° C. and 163.5° C. and 165.5° C., respectively, as well as a strong endotherm 184 at 194.8° C. and 196.8° C. Strong exothermic activity occurs at approximately 195° C. and 197° C.

Thus, in some embodiments, polymorph form C of decitabine may be characterizable by differential scanning calorimetry as having an endotherm between 44° C. and 54° C., an endotherm between 160° C. and 170° C., an endotherm between 190° C. and 200° C., and an exotherm between 190° C. and 200° C. More preferably, polymorph form C of decitabine may be characterizable by differential scanning calorimetry as having an endotherm between 47° C. and 52° C., an endotherm between 162° C. and 167° C., an endotherm between 190° C. and 195° C., and an exotherm between 193° C. and 198° C. More preferably, polymorph form C of decitabine may be characterizable by differential scanning calorimetry as having an endotherm between 48° C. and 50° C., an endotherm between 163° C. and 165° C., an endotherm between 191° C. and 193° C., and an exotherm between 194° C. and 196° C.

FIG. 18 shows a slight weight loss of approximately 1.2% at approximately 150° C. which is consistent with the moisture sorption/desorption analysis performed on form C illustrating that form C lost approximately 1.4% of its initial mass upon equilibrium at 5% RH. However, when the weight equilibrium event at 25° C. is omitted from the TGA method for a separate analysis of form C, a different result is obtained. See FIG. 29. In this case, the TGA plot for the sample displays a weight loss of approximately 3.2% at about 150° C. This result suggests that form C is an unstable hemi-hydrate polymorph of decitabine. A sample of form C prepared in the polymorph screen by vacuum evaporation of a solution of decitabine in water (sample no. 1029-65-05) is also found by TGA to contain a large amount of volatile material. For this sample, the weight loss is on the order of 7.2% at about 150° C., which is close to the theoretical loss of 7.3% predicted for the dehydration of decitabine monohydrate.

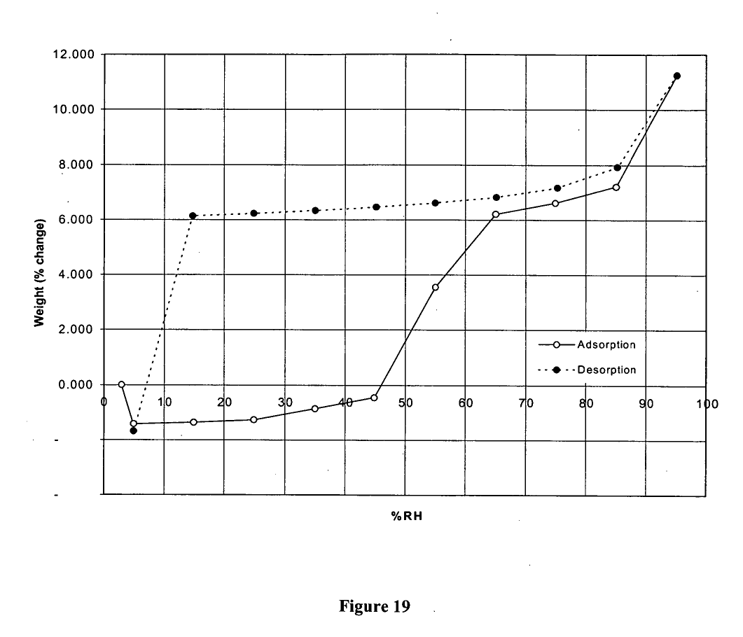

Moisture sorption/desorption data for Form C is provided below in Table 7 below and also in FIG. 19.

TABLE 7
Moisture Sorption/Desorption Data for Form B

| Elap Time | Weight | Weight | Samp Temp | |
|---|---|---|---|---|
| Min | Mg | % chg | deg C. | Samp RH % |
| 0.0 | 1.9540 | 0.0000 | 25.07 | 2.94 |
| 47.5 | 1.9263 | −1.4176 | 25.07 | 4.95 |
| 60.6 | 1.9274 | −1.3613 | 25.08 | 14.98 |
| 73.7 | 1.9292 | −1.2692 | 25.08 | 24.93 |
| 94.3 | 1.9371 | −0.8649 | 25.08 | 35.04 |
| 116.7 | 1.9451 | −0.4555 | 25.08 | 44.87 |
| 298.7 | 2.0233 | 3.5457 | 25.05 | 55.02 |
| 385.9 | 2.0754 | 6.2129 | 25.05 | 65.01 |
| 413.2 | 2.0834 | 6.6223 | 25.05 | 74.94 |
| 456.7 | 2.0950 | 7.2160 | 25.05 | 85.01 |
| 640.2 | 2.1739 | 11.2538 | 25.03 | 95.15 |
| 673.5 | 2.1088 | 7.9222 | 25.03 | 85.18 |
| 697.2 | 2.0942 | 7.1750 | 25.03 | 75.27 |
| 711.7 | 2.0875 | 6.8321 | 25.03 | 65.12 |
| 725.7 | 2.0833 | 6.6172 | 25.02 | 54.99 |
| 739.6 | 2.0802 | 6.4585 | 25.02 | 45.13 |
| 749.3 | 2.0779 | 6.3408 | 25.03 | 35.04 |
| 758.6 | 2.0757 | 6.2283 | 25.03 | 24.87 |
| 771.0 | 2.0739 | 6.1361 | 25.03 | 14.79 |
| 887.7 | 1.9212 | −1.6786 | 25.03 | 4.96 |

The sample losses 1.4% of its initial mass upon equilibrium at 5% RH, which is indicative of the presence of minor amounts of moisture in the sample. Form C is very hygroscopic as it absorbs close to 13% of its mass between 5% RH to 95% RH. The bulk of the mass loss for sample C occurred at the final RH event at 5% RH, which is similar to what was observed for form B. As the weight equilibrium is not met after 180 minutes at this RH level, the sample can absorb even more moisture if given longer time at this RH level. XRPD analysis of sample form C after moisture sorption/desorption analysis indicates that sample form C converts to form B.

These results agree with stress experiment data performed on form C using RH chambers. See Table 17 bottom two rows. Two of three samples converted from form C to form B upon storage at approximately 23% and 85% RH. The third sample of form C that was stored at approximately 33% RH remained unchanged after 28 days. These results suggest that form C may be converted to form B given enough time.

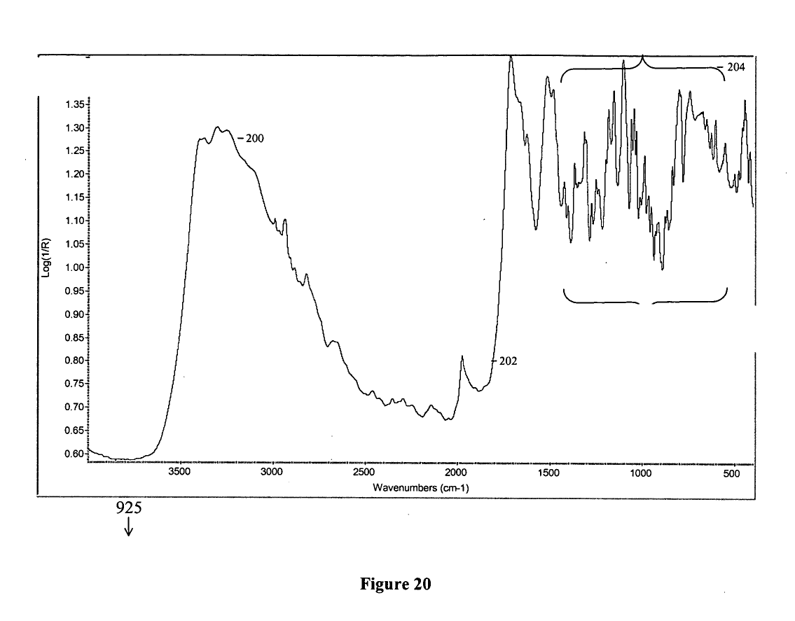

The IR spectrum for form C is provided in FIG. 20. The IR data collected on this form show a broad OH stretch 200 around 3400 cm⁻¹. A weak peak 202 at 2000 cm⁻¹, especially as compared to the sharp peak observed in polymorph forms A and B at 2000 cm⁻¹. A complex fingerprint morphology 204 between 1700 cm⁻¹ and 500 cm⁻¹ is also observed. Although each peak in the needle morphology corresponds with a peak in form A, the peaks are generally broader and longer. There are no absorption peaks observed between 3625 cm⁻¹, and 3675 cm⁻¹.

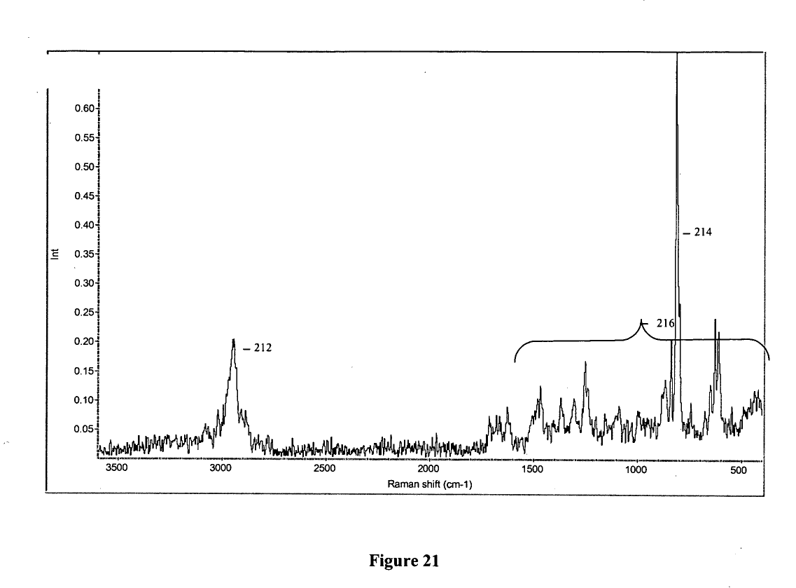

The Raman spectrum for form C is illustrated in FIG. 21 and shows weak peaks of aromatic and aliphatic CH stretches 202 between 3100 and 2800 cm⁻¹, a strong peak 204 at roughly 800 cm⁻¹, and weak bands 206 in the region of 600 cm⁻¹ to 1700 cm⁻¹.

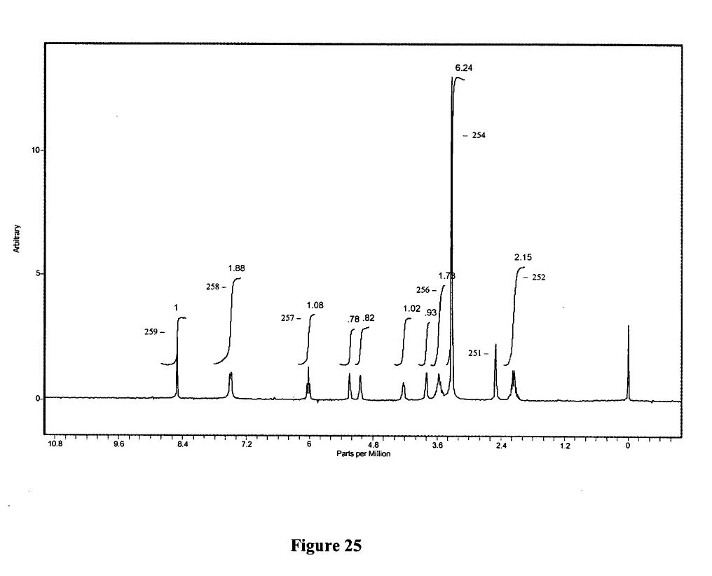

The ¹H NMR spectra of form C further illustrates that form C is a unique polymorph of decitabine. FIG. 25 provides the chemical shifts for decitabine polymorph form C. Shifts 258 and 259 indicate electronic properties of an aromatic ring. Shift 257 indicates a C═C double bond whereby the 257 peak is shorter than that of equivalent 247 peak polymorph form B. Shifts 254 and 256 indicate an ether region or a region of alcohols and esters. Peak 256 is substantially shorter than equivalent peak 246 in polymorph B. Chemical shifts 251 and 252 indicate a carbonyl region having protons attached to carbons next to a C═O, C═C or phenyl ring. Chemical shifts 251 and 252 are substantially

E. Formulations s and Administration Modalities

The present invention encompasses pharmaceutical formulations comprising one or more of the decitabine polymorphs disclosed herein. Such pharmaceutical formulations may furthermore include a carrier or diluent, wherein the decitabine remains in its polymorphic form.

Formulations according to the present invention may be adapted for any type of administration. For example, the formulations can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, intrathecally, or optionally in a slow release dosage form. In preferred embodiments, decitabine polymorphs are administered orally, by inhalation or by injection subcutaneously, intramuscularly, intravenously or directly into cerebrospinal fluid.

1. Oral and Parenteral Formulations

According to one embodiment, one or more of polymorphic forms disclosed herien may be formulated for oral administration. The concentration of the polymorphs given in any oral formulation is determined by the final desired formulation. The total amount of all polymorphs present in the formulation is preferably an amount that will allow a recommended dose to be conveniently administered. One factor in determining the amount of the polymorph or polymorphs contained in an oral dose is the required size of the delivery vehicle.

Solid dosage forms for oral administration include capsules, tablets, pills, powders, and granules. In solid dosage forms, the active agent is admixed with at least one inert pharmaceutically acceptable carrier such as sucrose, lactose, or starch. Such dosage forms can also comprise, as is normal practice, an additional substance other than an inert diluent, e.g., a lubricating agent such as magnesium stearate. With capsules, tablets, and pills, the dosage forms may also comprise a buffering agent. Tablets and pills can additionally be prepared with enteric coatings.

Liquid dosage forms for oral administration include pharmaceutically acceptable, suspensions and syrups, with the elixirs containing an inert diluent commonly used in the art, such as water. These compositions can also include one or more adjuvants, such as a surface stabilizing agent, a suspending agent, a sweetening agent, a flavoring agent or a perfuming agent. Decitabine is maintained in any disclosed polymorph form when the invention is embodied as a liquid dosage form.

According to this aspect, the decitabine polymorph is mixed with other compounds or delivery devices to form stable compositions with enhanced therapeutic activity. These formulations permit oral administration to tumor-bearing subjects, such as human patients with cancer. For example, in one embodiment, the decitabine polymorph forms may be mixed with pharmaceutically acceptable powdered excipients, carriers and/or diluents. The compositions and amount of each additional material in the formulation will depend upon various factors, including, the speed of administration, the timing of drug delivery after administration of the formulation and final desired concentration. Examples of excipients that may be included in such formulations include a pH adjustment compound, typically either a pharmaceutically acceptable acid or base, and/or a buffering agent, comprising approximately equimolar ratio of a weak acid or base and the conjugate salt thereof.

In one embodiment, the formulation may comprise a polymorph combined with a surface interaction inhibitor, which creates a physical barrier between adjacent particles. In this formulation, the decitabine is preferably a crystalline polymorph (e.g., a true solid) having a relatively small particle size, which is expected to stabilize the decitabine better than a glassy or amorphous, quasi-solid material having the same particle size. The small yet stable particles decitabine delivered in this composition are expected to have better bioavailability and higher therapeutic activity when administered orally compared to dosage forms having larger particle size, while having a longer shelf life than preparations comprising small glassy particles.

Preparations for parenteral administration include sterile aqueous or non-aqueous suspensions, and microsuspensions. Examples of non-aqueous vehicles are propylene glycol, polyethylene glycol, vegetable oils, such as olive oil and corn oil, gelatin, and injectable organic esters such as ethyl oleate. Those of skill in the art of formulating pharmaceutical preparations will appreciate that complete solvation of crystalline or amorphous solids is not encompassed by the instant invention and the polymorph should be insoluble in the carrier to preserve the polymorph that is to be employed in the specific formulation. Such dosage forms may also contain one or more adjuvants such as a preserving agent, for example a surface interaction inhibitor, a wetting agent and a dispersing agent. The dosage forms may be sterilized by, for example, filtration through a bacteria-retaining filter, by incorporating sterilizing agents into the compositions, by irradiating the compositions, or by heating the compositions. They can also be manufactured using sterile water, or some other sterile injectable medium, prior to use.

Pharmaceutical formulations for oral or parenteral administration may also comprise a decitabine polymorph-containing microsuspension, and may contain alternative pharmaceutically acceptable carriers, vehicles, additives, etc. particularly suited to oral or parenteral drug administration. Alternatively, a decitabine polymorph-containing microsuspension may be administered orally or parenterally without modification. Microsuspensions are thermodynamically stable dispersions of microcrystals, which may be stabilized by an interfacial film of surfactant molecules functioning as a dispersing agent (Encyclopedia of Pharmaceutical Technology (New York: Marcel Dekker, 1992), volume 9).

2. Pulmonary Administration

Any of the decitabine polymorphs herein may be employed for pulmonary administration. Both crystalline polymorphs, wherein the crystals are true solid materials, and wholly amorphous, glassy, quasi-solid polymorphs lend themselves to being rendered to an appropriate particle size for both dry and aerosolized liquid particle types of pulmonary delivery. The crystalline or glassy polymorphic forms of the decitabine is more stable over time than preparations wherein the decitabine molecules do not comprise a solid or quasi-solid, as when the decitabine molecules are solvated. By way of example rather than limitation, any crystalline polymorph decitabine can be used in a dry powder formulation for pulmonary delivery if it has been crystallized in microcrystalline form. Alternatively crystalline polymorphs of decitabine having may be ground or pulverized to obtain a sufficiently small particle size, which may render them a corresponding polymorph having increased amorphous content, or predominantly amorphous precipitate from rapid evaporation of solvent may be ground into a powdered glass form.

Dry powder formulations for pulmonary delivery include the crystalline or amorphous polymorph and any carrier suitable for pulmonary drug administration, although pharmaceutical sugars are generally preferred as carriers, e.g., fructose, galactose, glucose, lactitol, lactose, maltitol, maltose, mannitol, melezitose, myoinositol, palatinite, raffinose, stachyose, sucrose, trehalose, xylitol, and hydrates and combinations thereof. Selected components are initially combined and then blended to form a homogeneous, uniform powder mixture. Techniques for preparation of such powders are well known in the art; briefly, the preparation typically includes the steps of reducing the particle size of each component (as necessary), combining the individual components and blending. Techniques of reducing the particle size employ, by way of example, mills such as an air-jet mill or ball mill. Particle sizes having a diameter of between about 0.1 μm to about 65 μm are required for pulmonary administration. Blending methods include passing the combined powders through a sifter and blending the individual powders in a powder blender such as a “double cone” blender or a “V-blender.” Regardless of the specific technique employed the resulting powder must be both homogeneous and uniform. Typically, the active agents will make up from about 0.10% to about 99% (w/w) of the total formulation.

Pulmonary formulations of the present invention may also be administered as aerosol compositions. Aerosol formulations are known to those skilled in the art and described, for example, in Remington's Pharmaceutical Sciences, 19^{th} Ed. (Easton, Pa.: Mack Publishing Company, 1995). Briefly, the aerosol formulation of the invention is either a solution aerosol, in which the active agents are soluble in the carrier (e.g., propellant), or a dispersion aerosol, in which the active agents are suspended or dispersed throughout the carrier or carriers and optional solvent. In aerosol formulations, the carrier is typically a propellant, usually a liquefied gas or mixture of liquefied gases. For example, the carrier may be a fluorinated hydrocarbon. Preferred fluorinated hydrocarbons are selected from trichloromonofluoromethane, dichlorodifluoromethane, dichlorotetrafluoroethane, chloropentafluoroethane, 1-chloro-1,1-difluoroethane, 1,1,difluoroethane, octafluorocyclobutane, 1,1, 1,2-tetrafluoroethane (HFA-134a), 1,1,1,2,3,3,3-heptafluoropropane (HFA-227) and combinations thereof. As is readily appreciated by one skilled in the art, the aerosol formulations of the invention may include one or more excipients. The aerosol formulations may, for example, contain: an antioxidant (e.g., ascorbic acid) for inhibiting oxidative degradation of the active agents; a dispersing agent (e.g., sorbitan trioleate, oleyl alcohol, oleic acid, lecithin, corn oil, and combinations thereof) for preventing agglomeration of particles; and/or a lubricant (e.g., isopropyl myristate) for providing slippage between particles and lubricating the components, e.g., the valve and spring, of the inhaler.

As described with respect to the dry powder formulations, the particle size released from aerosol formulations must be appropriate for pulmonary administration. Solution aerosols inherently produce small particles upon actuation of the inhaler because the active agent is expelled along with the carrier, i.e., propellant, solution as it evaporates. Consequently, solution aerosol administration produces sufficiently small particles, e.g., within a range of about 0.1 μm to about 65 μm, of active agents. The crystalline and amorphous polymorphs of decitabine of the invention may only be delivered via aerosol as a dispersion of solid in a liquid carrier.

Dispersion aerosols contain undissolved active agents in which particle size remains constant, i.e., the size of the particles in the dispersion aerosol remains unchanged during delivery of the active agent. The active agents must therefore have an appropriate particle size before formulation into a dispersion aerosol. Thus, techniques for reducing the particle size of active agents as described above for the dry powder formulations are equally applicable for preparing active agents having an appropriate particle size in a dispersion aerosol. Further, the same ranges of particle sizes preferred for the dry powder formulations are applicable to dispersion aerosols.

Aerosol formulations of the invention may be prepared by utilizing a cold filling process. First, the components of the aerosol formulation and an aerosol container are cooled to about −40° C., so that the carrier, i.e., propellant, is a liquid. All the components except for the carrier are then placed into the aerosol container. Next, the carrier is added and the components are mixed. A valve assembly is then inserted into place. Finally, the valve assembly is crimped so that the container is airtight. The assembled container bearing the inhalant formulation may be allowed to return to ambient temperature after assembly. As an alternative to the cold filling process, the aerosol formulation may be prepared by transfer of a carrier from a bulk container after all the components except for the carrier are placed into an aerosol container and a valve assembly is then inserted and crimped into place. The liquid carrier is then metered under pressure through the valve assembly from a bulk container or tank. After the carrier is metered in, the container is checked to ensure that the pressurized contents do not leak. For both of these methods of preparing aerosol formulations, the active agent will typically make up from about 0.1 wt. % to about 40 wt. % of the total formulation. Preferably the active agents make up about 1 wt. % to about 15 wt. % of the total formulation.

The pulmonary formulations of the present invention may also be a liquid composition for inhalation, as is well known in the art. See, e.g., Remington: The Science and Practice of Pharmacy, supra. For the decitabine polymorphs of the instant invention, the liquid composition must be a microsuspension. Such liquid formulations include one or more carriers in addition to the active agents. As mentioned above, care must be taken that a carrier does not solvate the polymorph is employed. An example of a carrier is a sodium chloride solution having concentration making the formulation isotonic relative to normal body fluid. In addition to the carrier, the liquid formulations may contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof). Combining the components followed by conventional mixing effects a liquid formulation suitable for inhalation. Typically, the active agents will make up from about 0.01% to about 40% of the total formulation.

Various known devices may be used to administer pulmonary formulations, whether dry powder, aerosol or liquid. Dry powder inhalers are well known to those skilled in the art and are used to administer the aforementioned dry powder formulations. Suitable dry powder inhalation devices for administering the present formulations include, for example, TURBOHALER® (Astra Pharmaceutical Products, Inc., Westborough, Mass.), ROTAHALER® (Allen & Hanburys, Ltd., London, England). Aerosol formulations may be administered via pressurized metered-dose inhalers. Liquid formulations of the invention may be administered via a pump spray bottle or nebulizer.

Other active agents may also be included in the formulations of the invention, including other anti-proliferative, anti-neoplastic or anti-inflammatory or bronchodilating agents that dilate the airway and effect deeper delivery, especially for pathologies involving inflammation of the bronchi or alveoli, or airway obstruction, for example lung and broncoalveolar carcinomas. Agents that perform both these functions, such as long acting β adrenergic agonists, including salmeterol xinafoate, and phosphodiesterase inhibitors, including theophylline and other hypoxanthines, have been shown to exert a synergistic anti-inflammatory effect in inflammatory pathohysiologic processes in the lung by Pang et al. (2000) Am. J. Respir. Cell Mol. Biol. 23(1):79-85.

Examples of suitable additional active agents to be coadministered with decitabine in the treatment of proliferative respiratory disorders involving inflammation and/or obstruction include, without limitation, bronchodilators, including β adrenergic agonists, anticholinergics, phosphodiesterase inhibitors suitable for inhalation, and corticosteroids. Combinations of bronchodilators may also be used. Long acting β adrenergic agonists are particularly preferred, as they will not only provide anti-inflammatory effects that often important in treating neoplastic pathologies of the respiratory system, but may also effect deeper delivery into the lung; this is especially important for lung and bronchoalveolar carcinomas involving alveolar inflammation. Likewise, any glucocorticoid therapeutically suitable for administration by inhalant or a pharmaceutically suitable salt ester or other derivative thereof may be included for co-administration by inhalant.

As alluded to above, bronchodilators are useful to ensure delivery of active agent deep into the lungs. Typical bronchodilators of the anticholinergic type include, by way of example rather than limitation, atropinic compounds such as isatropium, which have been shown to be strongly synergistic (Dusser (1998) Ann. Fr. Anesth. Reanim. 17(Suppl. 2):40s-42s) with β agonists, specifically β₂ agonists, in bronchodilation for acute asthma and are expected to exert similar effects when used to open the airways to ensure deep delivery to the alveoli for delivery of anti-inflammatory agent. Typical bronchodilators of the β adrenergic agonist class include, but are not limited to, albuterol, bitolterol, clenbuterol, fenoterol, formoterol, levalbuterol (i.e., homochiral (R)-albuterol), metaproterenol, pirbuterol, procaterol, reproterol, rimiterol, salmeterol and terbutaline. The bronchodilator may be present in the formulation as a salt, ester, amide, prodrug, or other derivative, or may be functionalized in various ways as will be appreciated by those skilled in the art.

Other anti-inflammatory drugs can be combined with decitabine polymorphs. Corticosteroids and non-steroidal anti-inflammatory drugs (NSAIDS) are potential combinatorial therapy agents, and already used in the treatment of inflammatory airway disease and neoplasms in general. Cromolyn sulfate and the new class of leukotriene inhibitors are also used in treating inflammatory disease, and may therefore be employed in conjunction with the decitabine crystalline and amorphous polymorphs for inhalation therapy of both neoplasms associated with inflammation and primary inflammatory proliferative lung pathologies. Agents that are not primarily anti-inflammatory which have been evidenced to have anti-inflammatory activity include the long acting agonists and theophylline, as noted above, and macrolide antibiotics (Cazzola et al. (2000) Monaldi Arch. Chest Dis. 55(3):231-6), which include erythromycin and its derivatives, e.g., azithromycin and clarithromycin. Co-administration of antibiotics, including those with anti-inflammatory activity, or anti-viral agents, with the crystalline and amorphous polymorphs of the instant invention is desirable for treatment of pulmonary neoplasias, which predispose the lungs to infection, and for treating, proliferative inflammatory diseases of infectious etiology, such as pulmonary tuberculosis and viral pneumonitis.

3. Transdermal Administration

Particulate suspensions, microsuspensions and nano suspensions as well as emulsifications of various particulate sizes, including the particulate sizes appropriate for pulmonary administration may be converted to transdermal delivery of decitabine. Alternatively larger size crystalline and/or amorphous polymorphs of the invention may be formulated as an emulsified, including microemulsified, dispersion, with addition of an appropriate emulsifying agent. However the particulate sizes obtained for pulmonary administration may be directly combined with an appropriate agent that preserves the particles while permitting the diffusion of decitabine molecules there through and transdermally upon application through the skin.

F. Dosages

Useful dosages of polymorph(s) herein can be determined by comparing their in vitro activity, and in vivo activity in animal models. Methods for the extrapolation of effective dosages in mice, and other animals, to humans are known to the art. See, e.g., U.S. Pat. No. 4,938,949.

Generally, the concentration of polymorph(s) herein in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.005 to about 100 mg/kg of body weight per day, more preferably from about 0.1 to about 75 mg/kg of body weight per day, more preferably from about 0.3 to about 50 mg/kg of body weight per day, more preferably from about 0.6 to about 25 mg/kg of body weight per day, more preferably from about 1 to about 15 mg/kg of body weight per day, more preferably from about 2 to about 10 mg/kg of body weight per day, or more preferably from about 3 to about 5 mg/kg of body weight per day.

The compound may conveniently be administered in unit dosage form; for example, containing 0.05 to 1000 mg, conveniently 0.1 to 750 mg, most conveniently, 0.5 to 500 mg of active ingredient per unit dosage form.

Ideally, the active ingredient should be administered to achieve peak plasma concentrations of the active compound of from about 0.005 to about 75 μM, preferably, about 0.01 to 50 μM, most preferably, about 0.02 to about 30 μM. This may be achieved, for example, by the intravenous injection of a 0.0005 to 5% solution of the active ingredient, optionally in saline, or orally administered as a bolus containing about 0.01-1 mg of the active ingredient. Desirable blood levels may be maintained by continuous infusion to provide about 0.0001-5 mg/kg/hr or by intermittent infusions containing about 0.004-15 mg/kg of the active ingredient(s).

In some embodiments, one or more polymorphs are administered into a patient via an intravenous infusion. An intravenous infusion can be administered 1-24 hours per day, and the treatment can continue for approximately 1-100 days, more preferably for about 2-50 days, or more preferably for about 3-10 days. The dose administered per treatment can range from about 1-300 mg/m² more preferably from about 1-200 mg/m², more preferably from about 1-100 mg/m², more preferably from about 1-50 mg/m², more preferably from about 1-35 mg/m², more preferably from about 1-25 mg/m², more preferably from about 1-10 mg/m², more preferably from about 1-5 mg/m², more preferably from about 1-3 mg/m².

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or intravenous infusions.

G. Indications

The decitabine polymorphs may be used to treat any disease state in which decitabine is therapeutically effective. In order to take advantage of the novel polymorphs of the present invention, the pharmaceutical formulations in which the polymorphs are incorporated and administered to retain their polymorphic form.

According to one embodiment, a method is provided for treating a disease state comprising administering to a patient a formulation comprising one or more decitabine polymorphs.

In one variation, a formulation comprising a decitabine polymorph is administered to a patient having a disease state associated with an undesirable or uncontrolled cell proliferation. Such indications include, for example, restenosis (e.g., coronary, carotid, and cerebral lesions), benign tumors, various types of cancers such as primary tumors and tumor metastases, abnormal stimulation of endothelial cells (atherosclerosis), insults to body tissue due to surgery or other events leading to formation of scar tissue, abnormal wound healing, abnormal angiogenesis, diseases that produce fibrosis of tissue, repetitive motion disorders, disorders of tissues that are not highly vascularized, proliferative responses associated with organ transplants and various inflammatory proliferative diseases.

Generally, cells in a benign tumor retain their differentiated features and do not divide in a completely uncontrolled manner. A benign tumor is usually localized and nonmetastatic. Specific types of benign tumors that can be treated using the present invention include, without limitation, hemangiomas such as cavernous hemangioma, hepatocellular adenoma, cavernous hemangioma, focal nodular hyperplasia, acoustic neuromas, neurofibroma, bile duct adenoma, bile duct cystanoma, fibroma, lipomas, benign bone tumors, leiomyomas, mesotheliomas, teratomas, myxomas, nodular regenerative hyperplasia, trachomas and granulomatous inflammatory diseases both infectious, such as pyogenic granulomas, and non-infectious or idiopathic, such as sarcoidosis and berylliosis.

In a neoplasia such as a malignant tumor, cells become undifferentiated, do not respond to physiologic cell proliferation control signals, and multiply in an uncontrolled manner. The malignant tumor is invasive and capable of spreading to distant sites (metastasizing). Malignant tumors and other neoplasias may usually be divided into primary and secondary neoplasias. A primary neoplasia arises directly from the tissue of origin and may spread to contiguous tissues and organs by local invasion. A secondary neoplasia, or metastasis, is exemplified by a tumor that originated elsewhere in the body but has now spread to a distant organ. The common routes for spread of neoplasia are direct growth into adjacent structures, and metastatic spread through the vascular or lymphatic systems, and tracking along tissue planes and body spaces including peritoneal fluid, cerebrospinal fluid, etc.

Specific types of cancers or neoplasias, both primary and secondary, that can be treated using this invention include both carcinomas and sarcomas. Examples of specific carcinomas and sarcomas include leukemia, breast cancer, skin cancer, bone cancer, prostate cancer, liver cancer, lung cancer, neurological tumors of the brain, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteosarcoma, Ewing's sarcoma, reticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglioneuromas, hyperplastic corneal nerve tumor, marfanoid habitus tumor, Wilm's tumor, seminoma, ovarian tumor, leiomyomas, cervical dysplasia and other in situ carcinomas, neuroblastoma, retinoblastoma, soft tissue sarcoma, malignant carcinoid, topical skin lesion, mycosis fungoides, rhabdomyosarcoma, Kaposi's sarcoma, osteogenic and other sarcomas, malignant hypercalcemia, renal cell tumor, polycythemia vera, adenocarcinomas, glioblastoma multiforma, leukemias, lymphomas, melanoma, and epidermoid carcinomas.

Treatment of abnormal cell proliferation due to insults to body tissue during surgery may be possible for a variety of surgical procedures, including joint surgery, bowel surgery, and keloid scarring. Diseases that produce fibrotic tissue include emphysema. Repetitive motion disorders that may be treated using the present invention include carpal tunnel syndrome.

The proliferative responses associated with organ transplantation that may be treated using this invention include those proliferative responses contributing to potential organ rejections or associated complications. Specifically, these proliferative responses may occur during transplantation of the heart, lung, liver, kidney, and any foreign or non-self cells, tissues, organs or organ systems.

Abnormal angiogenesis that may be may be treated using this invention include those abnormal angiogenesis accompanying rheumatoid arthritis, ischemic-reperfusion related brain edema and injury, adrenal cortical ischemia, ovarian hyperplasia and hypervascularity, polycystic ovary syndrome, endometriosis, psoriasis, diabetic retinopaphy, and other ocular angiogenic diseases such as retinopathy of prematurity (retrolental fibroplastic disease), macular degeneration, corneal graft rejection, neuroscular glaucoma and Oster Webber syndrome.

Diseases associated with abnormal angiogenesis require or induce vascular growth. For example, corneal angiogenesis involves three phases: a pre-vascular latent period, active neovascularization, and vascular maturation and regression. The identity and mechanism of various angiogenic factors, including elements of the inflammatory response, such as leukocytes, platelets, cytokines, and eicosanoids, or unidentified plasma constituents have yet to be revealed.

In another embodiment of the present invention, a method is provided for treating diseases associated with undesired and uncontrolled angiogenesis. The method comprises administering to a patient suffering from uncontrolled angiogenesis a therapeutically effective amount of a decitabine polymorph disclosed herein, such that formation of blood vessels is inhibited. The particular dosage of decitabine required to inhibit angiogenesis and/or angiogenic diseases may depend on the severity of the condition, the route of administration, and related factors that can be decided by the attending physician. Generally, accepted and effective daily doses are the amount sufficient to effectively inhibit angiogenesis and/or angiogenic diseases.

According to this embodiment, the composition of the present invention may be used to treat a variety of diseases associated with uncontrolled angiogenesis such as retinal/choroidal neovascularization and corneal neovascularization. Examples of retinal/choroidal neovascularization include, without limitation, Best's disease, myopia, optic pits, Stargart's disease, Paget's disease, vein occlusion, artery occlusion, sickle cell anemia, sarcoid, syphilis, pseudoxanthoma elasticum carotid abostructive diseases, chronic uveitis/vitritis, mycobacterial infections, Lyme disease, systemic lupus erythematosis, retinopathy of prematurity, Eale's disease, diabetic retinopathy, macular degeneration, Behcet's disease, infections causing a retinitis or choroiditis, ocular histoplasmosis, pars planitis, chronic retinal detachment, hyperviscosity syndromes, toxoplasmosis, trauma and post-laser complications, diseases associated with rubesis (neovascularization of the angle) and diseases caused by the abnormal proliferation of fibrovascular or fibrous tissue including all forms of proliferative vitreQretinopathy. Examples of corneal neuvascularization include, but are not limited to, epidemic keratoconjunctivitis, Vitamin A deficiency, contact lens overwear, atopic keratitis, superior limbic keratitis, pterygium keratitis sicca, Sjogren's syndrome, acne rosacea, phylectenulosis, diabetic retinopathy, retinopathy of prematurity, corneal graft rejection, Mooren ulcer, Terrien's marginal degeneration, marginal keratolysis, polyarteritis, Wegener granulomatosis, sarcoidosis, scleritis, pemphigoid, radial keratotomy, neovascular glaucoma and retrolental fibroplasia, syphilis, Mycobacteria infections, lipid degeneration, chemical bums, bacterial ulcers, fungal ulcers, Herpes simplex infections, Herpes zoster infections, protozoan infections and Kaposi sarcoma.

In yet another embodiment of the present invention, a method is provided for treating chronic inflammatory diseases associated with uncontrolled angiogenesis. The method comprises administering to a patient suffering from a chronic inflammatory disease associated with uncontrolled angiogenesis a therapeutically effective amount of the composition of the present invention, such that formation of blood vessels is inhibited. The chronic inflammation depends on continuous formation of capillary sprouts to maintain an influx of inflammatory cells. The influx and presence of the inflammatory cells produce granulomas and thus maintains the chronic inflammatory state. Inhibition of angiogenesis using the composition of the present invention alone or in conjunction with other anti-inflammatory agents may prevent the formation of the granulomas, thereby alleviating the disease. Examples of chronic inflammatory disease include, but are not limited to, inflammatory bowel diseases such as Crohn's disease and ulcerative colitis, psoriasis, sarcoidosis, and rheumatoid arthritis.

Inflammatory bowel diseases such as Crohn's disease and ulcerative colitis are characterized by chronic inflammation and angiogenesis at various sites in the gastrointestinal tract. For example, Crohn's disease occurs as a chronic transmural inflammatory disease that most commonly affects the distal ileum and ascending colon but may also occur in any part of the gastrointestinal tract from the mouth to the anus and perianal area. Patients with Crohn's disease generally have chronic diarrhea associated with abdominal pain, fever, anorexia, weight loss and abdominal swelling. Ulcerative colitis is also a chronic, nonspecific, inflammatory and ulcerative disease arising in the colonic mucosa and is characterized by the presence of bloody diarrhea.

These inflammatory bowel diseases are generally caused by chronic granulomatous inflammatory pathophysiologic processes. Inflammatory bowel disease may affect the entire gastrointestinal tract, typically involving new capillary sprouts surrounded by a cylinder of inflammatory cells. Inhibition of angiogenesis by the composition of the present invention should inhibit the formation of the sprouts and prevent the formation of granulomas. The inflammatory bowel diseases also exhibit extra intestinal manifestations, such as skin lesions. Such lesions are characterized by inflammation and angiogenesis and can occur at many sites other the gastrointestinal tract. Inhibition of angiogenesis by the composition of the present invention should reduce the influx of inflammatory cells and prevent, halt or slow pathogenesis of the lesion.

Sarcoidois, another chronic inflammatory disease, is characterized as an idiopathic multisystem granulomatous disorder. Berylliosis resembles sarcoidosis histopathologically, but is known to be caused by the element Beryllium. The granulomas of sarcoidosis and berylliosis histopathologically resemble the non-caseating granulomas of Mycobacterium tuberculosis and other diseases caused by Mycobacteria, but caseating granulomas found in M. Tuberculosis infection are absent in both berylliosis and sarcoidosis. The granulomas of this disease can form anywhere in the body and, thus, the symptoms depend on the site of the granulomas and whether the disease is active. The formation of sarcoid granulomas is facilitated by the angiogenic capillary sprouts, which provide a constant supply of inflammatory cells. By using the composition of the present invention to inhibit angiogenesis, such granuloma formation can be inhibited.

Psoriasis, also a chronic and recurrent inflammatory disease, is characterized by papules and plaques of various sizes. Treatment using the composition of the present invention alone or in conjunction with other anti-inflammatory agents should prevent the formation of new blood vessels necessary to maintain the characteristic lesions and provide the patient relief from the symptoms.

Rheumatoid arthritis (RA) is also a chronic inflammatory disease characterized by non-specific inflammation of the peripheral joints. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis. Treatment using the composition of the present invention alone or in conjunction with other anti-RA agents should prevent the formation of new blood vessels necessary to maintain the chronic inflammation and provide the RA patient relief from the symptoms.

The composition of the present invention may also be used in conjunction with other anti-angiogenesis agents to inhibit undesirable and uncontrolled angiogenesis. Examples of anti-angiogenesis agents include, but are not limited to, retinoic acid and derivatives thereof, 2-methoxyestradiol, ANGIOSTATIN™ protein, ENDOSTATIN™ protein, suramin, squalamine, tissue inhibitor of metalloproteinase-I, tissue inhibitor of metalloproteinase-2, plasminogen activator inhibitor-1, plasminogen activator inhibitor-2, cartilage-derived inhibitor, paclitaxel, platelet factor 4, protamine sulphate (clupeine), sulphated chitin derivatives (prepared from queen crab shells), sulphated polysaccharide peptidoglycan complex (sp-pg), staurosporine, modulators of matrix metabolism, including for example, proline analogs ((1-azetidine-2-carboxylic acid (LACA), cishydroxyproline, d-1,3,4-dehydroproline, thiaproline], α,α-dipyridyl, β-aminopropionitrile fumarate, 4-propyl-5-(4-pyridinyl)-2(3h)-oxazolone; methotrexate, mitoxantrone, heparin, interferons, 2 macroglobulin-serum, chimp-3, chymostatin, β-cyclodextrin tetradecasulfate, eponemycin, fumagillin, gold sodium thiomalate, d-penicillamine (CDPT), β-1-anticollagenase-serum, α-2-antiplasmin, bisantrene, lobenzarit disodium, n-(2-carboxyphenyl-4-chloroanthronilic acid disodium or “CCA”, thalidomide; angiostatic steroid, carboxyaminoimidazole; metalloproteinase (metalloprotease) inhibitors such as BB94. Other anti-angiogenesis agents include antibodies, preferably monoclonal antibodies against these angiogenic growth factors: bFGF, aFGF, FGF-5, VEGF isoforms, VEGF-C, HGF/SF and Ang-1/Ang-2. Ferrara N. and Alitalo, K. “Clinical application of angiogenic growth factors and their inhibitors” (1999) Nature Medicine 5:1359-64.

In all embodiments, the term “effective amount” is understood as a medical art term, that is, the dose schedule and route of administration of the drug that gives the best therapeutic value and convenience to the patient.

### EXAMPLES

### Example 1

A sample of decitabine used for the polymorph screen was provided by SuperGen Inc. A representative XRPD pattern exhibited by this ample is provided in FIG. 1. The polymorph form of decitabine exhibiting this pattern is designated as form A. Form A is thermally stable but will readily hydrate to form B upon exposure to water or atmospheric moisture. Form B will convert to either form A or form C depending on the experimental conditions. Form C readily converts to form B in the presence of atmospheric moisture such that it is difficult to obtain a pure sample of form C in the laboratory.

The samples prepared for the polymorph screen of decitabine were classified according to similar XRPD patterns. One XRPD pattern from a series of matching patterns was designated as the “standard” pattern, which was then used for future comparisons. Amorphous samples are identified by the absence of well-defined peaks and the presence of a broad “halo” feature in the XRPD pattern of the sample. Disordered material is characterized by broad peaks in the XRPD pattern of the sample. Solution ¹H NMR spectroscopy was used to verify that each solid form is indeed a solid modification of decitabine, and not a decomposition product. ¹H NMR spectroscopy for polymorphs A, B and C is illustrated in FIGS. 23-25, respectively.

### Example 2

A weighed sample of decitabine (typically 10 to 20 mg) was treated with aliquots of the test solvent. Solvents were either reagent or HPLC grade. The aliquots were typically either 100 μL or 1 mL. Between additions, the mixture was typically shaken or sonicated. Whether the solids dissolved was judged by visual inspection. Solubilities were estimated from these experiments based on the total solvent used to provide complete dissolution. The approximate solubilities of decitabine in various solvents are provided in Table 15 below.

TABLE 15
Approximate Solubility of Decitabine

| Solvent | Solubility (mg/mL) | Sample No. |
|---|---|---|
| Acetone | <1 | 1029-09-04 |
| Acetonitrile | <1 | 1029-11-04 |
| Acetonitrile:Water (1:1) | 22 | 1029-67-03 |
| 2-Butanone | <1 | 1029-11-06 |
| Chloroform | <1 | 1029-09-01 |
| Dichloromethane | <1 | 1029-11-05 |
| Dichloromethane:Ethanol (1:1) | <1 | 1029-29-07 |
| Dichloromethane:Methanol (1:1) | >1 | 1029-29-06 |
| Diethylamine | <1 | 1029-56-01 |
| N,N-Dimethylformamide | 5 | 1029-68-01 |
| 1,4-Dioxane | <2 | 1059-59-01 |
| Ethanol:Water (1:1) | 3 | 1029-29-05 |
| Ethyl Acetate | <1 | 1029-11-02 |
| Ethyl Ether | <1 | 1029-11-01 |
| 1,1,1,3,3,3-Hexafluoro-2-propanol | 18 | 1029-62-06 |
| Hexanes | <1 | 1029-09-06 |
| Methanol | 2 | 1029-09-03 |
| Methanol:2,2,2-Trifluoroethanol (1:1) | >1 | 1029-29-03 |
| Methanol:Water (1:1) | 4 | 1029-29-04 |
| Methyl Sulfide | <1 | 1029-68-02 |
| Methyl Sulfoxide | 37 | 1029-66-01 |
| Nitromethane | <1 | 1029-56-03 |
| 2-Propanol | <1 | 1029-11-03 |
| Tetrahydrofuran | <1 | 1029-09-05 |
| Toluene | <1 | 1029-67-05 |
| 1,1,1-Trichloroethane | <1 | 1029-56-02 |
| 2,2,2-Trifluoroethanol | 2 | 1029-37-03 |
| 2,2,2-Trifluoroethanol:Water (9:1) | 5 | 1029-29-02 |
| Water | 8 | 1029-09-02 |

Solubilities were estimated from these experiments based on the total solvent used to give a solution. Duplicate runs were averaged. The actual solubilities may be greater than those calculated due to the size of the solvent aliquots used, or due to a slow rate of dissolution. If dissolution did not occur during the experiment the solubility is expressed as “less than.”

In general, decitabine is poorly soluble in almost all the solvents used in this study. The notable exception is methyl sulfoxide, in which the compound was found be soluble to the extent of approximately 37 mg/mL. Decitabine is also slightly soluble in 1,1,1,3,3,3-hexafluoro-2-propanol (˜18 mg/mL) and sparingly soluble in water (˜8 mg/mL).

### Example 3

Solutions were filtered using one of several different final processing steps. Such processing steps include: fast evaporation, slow evaporation, centrifugal evaporation under reduced pressure, slow cool, solvent/anti-solvent crash, crash cool, slurry experiments, relative humidity (RH) stress, elevated temperature slurry experiments, vapor diffusion, milling experiments, and lyophilization (freeze drying).

In fast evaporation (FE), a solution of decitabine was prepared in a given solvent and filtered through a 0.2-μm nylon filter. The filtered solution was allowed to evaporate at ambient temperature in an open vial.

In slow evaporation (SE), a solution of decitabine was prepared in a given solvent and filtered through a 0.2-μm filter. The filtered solution was allowed to evaporate at ambient temperature in a vial that was either capped loosely or covered with a piece of aluminum foil containing pinholes.

In centrifugal evaporation under reduced pressure (CentriVap), a solution of decitabine was prepared in a given solvent and filtered through a 0.2-μm filter into a vial. The vial was then placed in a Labconco CentriVap® centrifugal evaporator and the solvent was removed under reduced pressure using a mechanical vacuum pump to provide a solid residue.

In slow cool (SC), a solution of decitabine was prepared in a given solvent and heated on a hot plate that was typically set to a nominal temperature of 65° C. The solution was filtered through a 0.2-μm filter into open vial while still warm. The vial was sealed and allowed to cool slowly to ambient temperature. The presence or absence of solids was noted. If there were no solids present, or if the amount of solids was judged to be too small for XRPD analysis, the vial was placed in a refrigerator overnight. Again, the presence or absence of solids was noted and if there were insufficient solids the vial was placed in a freezer overnight. If insufficient solids were still present, the solution was allowed to evaporate at ambient temperature with the cap for the sample vial loosened. In this case the samples are noted as SC, SE. Solids that formed were isolated by filtration and allowed to air-dry prior to analysis.

In solvent/anti-solvent crash (S/AS), solutions of decitabine were prepared in various solvents and filtered through a 0.2-μm filter. Solid formation was induced by adding the filtered solution to an appropriate anti-solvent at a given temperature. The resulting solids were isolated by filtration and air-dried prior to analysis. In cases where no solids formed immediately, the samples were placed in a freezer or refrigerator to facilitate crystallization. If no solids formed, the solution was allowed to evaporate at ambient temperature with the cap for the sample vial loosened. In these cases the samples are noted as S/AS, SE.

In crash cool (CC), solutions of decitabine were prepared in various solvents and filtered through a 0.2-μm filter. Solid formation was induced by adding the filtered solution to a vial and immediately placing the sample into a dry ice/acetone bath for several minutes. The resulting solids were isolated by filtration and air-dried prior to analysis. In cases where no solids formed immediately, the samples were placed in a freezer to facilitate crystallization.

In slurry experiments, enough decitabine was added to a given solvent so that undissolved solids were present. The mixture was then agitated in a sealed vial at ambient temperature using either an orbital shaker or a rotating wheel. After several days the solids were isolated by vacuum filtration and allowed to dry at ambient temperature with the cap for the sample vial loosened.

In vapor diffusion (VD), open vials containing a solution of decitabine that was prepared in a given solvent and filtered through a 0.2-μm nylon filter were placed inside a larger vial containing solvent. The larger vial was sealed and allowed to stand at ambient temperature for several days. In milling experiments, samples of decitabine were ground either at room temperature using a ball mill (Retsch Mixer Mill model MM200) or at liquid nitrogen temperatures using a cryogrinder (SPEX CertiPrep Model 6750 Freezer/Mill).

In elevated temperature slurry experiments, solutions of decitabine were prepared by adding enough solids to a given solvent so that undissolved solids were present. The mixture was then agitated in a sealed vial at elevated temperature using an orbital shaker. After several days the solids were isolated by suction filtration and allowed to dry at ambient temperature with the cap for the sample vial loosened.

Results of decitabine polymorph screen are summarized in Table 16 below.

TABLE 16
Decitabine Polymorph Screen

| | | XRPD |
|---|---|---|
| Solvent | Method | Pattern^{a} |
| (none) | Hydraulic Press 10,000 lbs | A + 1 peak |
| | ˜3 min | |
| | Hydraulic Press 10,000 lbs | A |
| | 1 hour | |
| | Form B Post TGA @ 150° C. | A |
| | 10 min | |
| | B (PO) ground w/mortar | B |
| | and pestle (2 min) | |
| | Form C stored @ ambient | B |
| | 43 days | |
| Acetone | Slurry (Ambient); 12 | A |
| | days, vacuum oven dried | |
| | (ambient) | |
| | Slurry (50° C.); 12 days, | A |
| | vacuum oven dried | |
| | (ambient) | |
| 2-Butanone | Slurry (ambient); 12 days, | A |
| | vacuum oven dried | |
| | (ambient) | |
| | Slurry (50° C.); 12 days, | A + 1 peak |
| | vacuum oven dried | |
| | (ambient) | |
| Chloroform | Slurry (ambient) | A + 1 peak |
| | 12 days, vacuum | |
| | oven dried (ambient) | |
| | Slurry (50° C.) | A |
| | 12 days, vacuum | |
| | oven dried (ambient) | |
| Dichloromethane | Slurry (ambient); 12 days, | A (PO) |
| | vacuum oven dried | |
| | (ambient) | |
| Dichloromethane:Methanol | Slurry (ambient) -> FE | B (PO) |
| (1:1) | | |
| Ethyl Acetate | Slurry (50° C.); 12 days, | A |
| | vacuum oven dried | |
| | (ambient) | |
| | Slurry (ambient) | A |
| | 12 days, vacuum | |
| | oven dried (ambient) | |
| 1,2- | Slurry (ambient) | B + 1 peak |
| Dimethoxyethane | 27 days | |
| | Forms A/C | B |
| | Slurry (ambient) 27 days | |
| Ethyl Ether | Slurry (ambient) 12 days, | A |
| | vacuum oven dried | |
| | (ambient) | |
| 1,1,1,3,3,3-Hexafluoro- | FE | B (PO) |
| 2-propanol | SC, SE | B |
| | | B (PO) |
| | SE | B (PO) |
| Hexanes | Slurry (ambient) | A (PO) |
| | 12 days, vacuum | |
| | oven dried (ambient) | |
| Methanol | CC, vacuum | A |
| | oven dried (ambient) | |
| | FE | B + 1 peak |
| | | B (PO) |
| | Forms B/C slurry | A |
| | (ambient) 10 days | |
| | SC | B |
| | SC vacuum oven dried | A (PO) |
| | (ambient) | |
| Methanol:2,2,2- | Slurry (ambient) -> FE | B + 1 peak |
| Trifluoroethanol (1:1) | | |
| Methyl Sulfide | Slurry (ambient) | A |
| | 12 days, vacuum | |
| | oven dried (ambient) | |
| 2-Propanol | Slurry (ambient) | A |
| | 12 days, vacuum | |
| | oven dried (ambient) | |
| | Slurry (50° C.) | A (PO) |
| | 12 days, vacuum | |
| | oven dried (ambient) | |
| 1,1,1-Trichloethane | Slurry (ambient) | A |
| | 9 days, vacuum | |
| | oven dried (ambient) | |
| 2,2,2-Trifluoroethanol | CC, FE | B |
| 2,2,2-trifluoroethanol:Water | CC | B |
| (9:1) | FE | Disordered B |
| | CentriVap (ambient); | C |
| | vacuum oven dried | C |
| | (ambient) | |
| Water | CentriVap (ambient) | B |
| | vacuum oven dried | B |
| | (ambient) | |
| | FE, vacuum oven dried | B |
| | (ambient) | |
| | Freeze dried | B |

^{a}PO = Preferred Orientation

### Example 4

In relative humidity (RH) stress analysis, open vials containing solid samples were placed inside chambers containing saturated salt solutions along with a small amount of the undissolved salt. The chambers were sealed and allowed to stand at ambient temperature for several days. Samples were analyzed by X-ray powder diffraction (XRPD) immediately after removing the sample from the RH chamber. The RH values these salt solutions were obtained from an ASTM standard. RH results are illustrated in Table 17 below:

TABLE 17
Relative Humidity Stress Experiments

| | RH Condition | | Stress | |
|---|---|---|---|---|
| Initial | Aqueous Salt | % RH | Period | XRPD |
| Form^{a} | Solution | @ 20° C.^{b} | (days) | result |
| A | Potassium Acetate | 23.1 | 27 | A |
| A | Magnesium Chloride | 33.1 | 27 | A |
| A | Potassium Carbonate | 43.2 | 27 | A |
| A | Sodium Chloride | 75.5 | 27 | B |
| A | Sodium Chloride | 75.5 | 27 | B |
| A | Potassium Chloride | 85.1 | 27 | B |
| C | Potassium Acetate | 23.1 | 25 | B |
| C | Magnesium Chloride | 33.1 | 28 | C |
| C | Potassium Chloride | 85.1 | 28 | B |

Finally in lyophilization (freeze drying), solutions were frozen in a dry ice/acteone bath and then placed on a commercial freeze dryer equipped with a rotary vane mechanical vacuum pump. No attempt was made to control the temperature of the frozen solution during the freeze drying operation.

Hygroscopicity was investigated by placing a sample in a sealed chamber at room temperature and 95% relative humidity for 20 days. Weight gain/loss or TGA were not measured in the course of this study of hygroscopicity. An XRPD pattern was obtained on the solid remaining after 20 days and compared to the starting material.

Dehydration/desolvation studies were conducted by placing a sample under continuous vacuum at room temperature for 14 days. An XRPD pattern was obtained on the remaining solid and compared to the starting material.

A solidified melt of decitabine was produced by slowly heating the sample on a hot bench until a visual melt was observed and then quickly cooling the sample to ambient temperature. As the material began to melt, it turned dark and bubbled. The resulting dark material was not analyzed further due to decomposition.

### Example 5

### Single Crystal Growth

A solution was prepared by almost dissolving 35.5 mg of form A in 4.0 mL of methanol that was heated on a hot plate set to 100° C. (The temperature of the methanol was 55° C.) The solution was filtered into a vial, which was then sealed and allowed to cool to ambient temperature. Solids formed overnight. Several crystals were placed onto a microscope slide and protected with Paratone-N.

A colorless plate of C₈H₁₂N₄O₄ having approximate dimensions of 0.28×0.25×0.05 mm was mounted on a glass fiber in random orientation. Preliminary examination and data collection were performed with Mo K_{α} radiation (λ=0.71073 Å) on a Nonius KappaCCD diffractometer. Refinements were performed on an Alphaserver 2100 using SHELX97. The crystallographic drawing of the asymmetric unit was obtained using the program ORTEP and packing diagrams were generated using Mercury ver. 1.1 software.

Cell constants and an orientation matrix for data collection were obtained from least-squares refinement using the setting angles of 4960 reflections in the range 2<θ<25°. The orthorhombic cell parameters and calculated volume are: a=5.6268 (2), b=7.0943 (2), c=24.8394 (10) Å, α=β=γ=90°, V=991.54 (6) Å³. For Z=4 and a molecular weight of 228.21 the calculated density is 1.53 g cm⁻³. The refined mosaicity from DENZO/SCALEPACK was 0.42° indicating good crystal quality. The space group was determined, by the program ABSEN, from the systematic presence of:
h00 h=2n
0k0 k=2n
00l l=2n
   and from subsequent least-squares refinement, and determined to be P212121 (no. 19). The data were collected to a maximum 2θ value of 50.0°, at a temperature of 150±1 K.

The crystallographic data for this structure includes a molecular formula of C₈H₁₂N₄O₄, molecular weigh of 228.21 and a space group of P2₁2₁2₁. The quality of the structure obtained is high, as indicated by the R-value of 0.033 or 3.3%. The asymmetric unit contains only one, symmetry independent, molecule. See FIG. 6. The crystal packing of form A is characterized by a corrugated tape structure that forms as a result of hydrogen bonding between the azocytosine rings. FIG. 7. The one-dimensional tape units then stack in corrugated layers that are joined together by relatively weak hydrogen bonds between the deoxyribose rings. FIG. 8. The calculated XRPD pattern from the single crystal X-ray data is given FIG. 1. Comparison of this calculated pattern with the experimental XRPD pattern for form A provides an excellent match between the two data sets.

### Example 6

### Characterization

A. X-Ray Powder Diffraction

X-ray powder diffraction analyses were carried out on a Shimadzu XRD-6000 X-ray powder diffractometer using Cu Kα radiation having a wavelength of 1.5406 Å. The instrument is equipped with a fine-focus X-ray tube. The tube power was set by setting potential difference at 40 kV, and current 40 mA. The divergence and scattering slits were set at 1° and the receiving slit was set at 0.15 mm. Diffracted radiation was detected by a NaI scintillation detector. A theta-two theta continuous scan at 3°/min (0.4 sec/0.02° step) from 2.5 to 40 °2θ was performed. A silicon standard was analyzed each day to check the instrument alignment. Each sample was analyzed in a quartz sample holder. A variable temperature (VT-XRPD) experiment was performed on one form. The sample was prepared for analysis by pressing it into a variable temperature holder.

B. Thermo and Thermogravimetric Analysis

Thermogravimetric analysis (TGA) was carried out on TA Instruments TGA 2050 or 2950. The calibration standards were nickel and Alumel™. Samples were placed on a clean, aluminum sample pan, accurately weighed, and inserted into the TGA furnace. The samples were heated in nitrogen at a rate of 10° C./min, from 35° C. to a final temperature of 250° C.

Differential scanning calorimetry (DSC) data were obtained on a TA Instruments DSC 2920. The calibration standard was indium. Samples were placed into a DSC pan, and the weight accurately recorded. The pans were either crimped pans or hermetically sealed pans with a pinhole to allow for pressure release. Note that the observed volatilization temperatures may be higher than those obtained in open pans due to pressure effects.

The samples were heated under nitrogen at a rate of 10° C. min, from 25° C. to a final temperature of either 250° C. or 350° C.

C. Hot-stage Microscopy

Hot-stage microscopy was carried out using a Linkam hot stage (model FT IR 600) apparatus mounted on a Leica DM LP Microscope equipped with a Sony DVC-970MD 3CCD camera for collecting images. A 20× objective was used with cross polarizers to view samples. The stage temperature was calibrated using USP standards each day prior to running samples. For each sample, a small quantity was placed on a microscope slide and covered and a drop of silicon oil was added on the solid. Samples were heated at approximately 4° C./min. and images were captured periodically using the 20× objective lens and a CCD camera. A cross-polarizing filter was used to observe birefringence.

D. Infrared (IR) Spectroscopy

IR spectra were acquired on a Magma™ model 860 Fourier transform IR spectrophotometer from Nicolet Instrument Corp. equipped with an Ever-Glo mid/far IR source, an extended range potassium bromide (KBr) beamsplitter, and deuterated triglycine sulfate (DTGS) detector. A Spectra-Tech, Inc. diffuse reflectance accessory (the Collector™) was utilized for sampling. Each spectrum represents 256 co-added scans at a spectral resolution of 4 cm⁻¹. Sample preparation for the compound consisted of placing the sample into a microcup and leveling the material with a frosted glass slide. A background data set was acquired with an alignment mirror in place. A single beam sample data set was then acquired. Subsequently, a Log 1/R (R=reflectance) spectrum was acquired by taking the ratio of the sample single-beam data set to the background single beam data set. The spectrophotometer wavelength was calibrated with polystyrene prior to the time of use.

E. Raman Spectroscopy

FT-Raman spectra were acquired on an FT-Raman 960 spectrometer (Thermo Nicolet) utilizing an excitation wavelength of 1064 nm and approximately 0.5 W of Nd:YVO₄ laser power. The Raman Spectra were measured with an indium gallium arsenide (InGaAs) detector. Each sample was prepared for analysis by placing it in a solid holder. A total of 256 sample scans were collected at a spectral resolution of 4 cm⁻¹. The spectrometer was calibrated (wavelength) with sulfur and cyclohexane at the time of use.

F. NMR Spectroscopy

Solution state ¹H NMR spectra were obtained at ambient temperature on a Bruker model AM-250 spectrometer operating at 5.87 T (Larmor frequency: ¹H=250 MHz). Time-domain data were acquired using a pulse width 7.5 μs and an acquisition time of 1.6384 second over a spectral window of 5000 Hz. A total of 16384 data points were collected. A relaxation delay time of 5 seconds was employed between transients. Each data set typically consisted of 128 co-averaged transients. The spectra were processed utilizing GRAMS/32 AI software, version 6.00. The free induction decay (FID) was zero-filled to four times the number of data points and exponentially multiplied with a line-broadening factor of 0.61 Hz prior to Fourier transformation. The ¹H spectra were internally referenced to tetramethylsilane (0 ppm) that was added as an internal standard.

G. Moisture Balance

Moisture sorption/desorption data were collected on a VTI SGA-100 Vapor Sorption Analyzer. Sorption and desorption data were collected over a range of 5 to 95% relative humidity (RH) at 10% RH increments under a nitrogen purge. Sodium chloride (NaCl) and polyvinylpyrrolidone (PVP) were used as the calibration standards. Equilibrium criteria used for analysis were less than 0.0100% weight change in 5 minutes, with a maximum equilibration time of 180 minutes if the weight criterion was not met. Data collected were not corrected for the initial moisture content of the samples.

It will be apparent to those skilled in the art that various modifications and variations can be made to the compounds, compositions, and methods of the present invention without departing from the spirit or scope of the invention. Thus, it is intended that the present invention cover the modifications and variations of this invention provided they come within the scope of the appended claims and their equivalents.

### Example 7

### Amorphous Material

Amorphous material was prepared by crystallizing decitabine from water (sample no. 1029-39-04).

### Example 8

In VT-XRPD experiments, decitabine polymorph form B converted to a mixture of forms B and C, while in an experiment performed in the TGA furnace form B converted to form A at about 150° C. The only other difference in these two experiments apart from sample size is that in the VT-XRPD experiment the sample is heated in the presence of air while in the TGA experiment dry nitrogen is used.

### Example 6

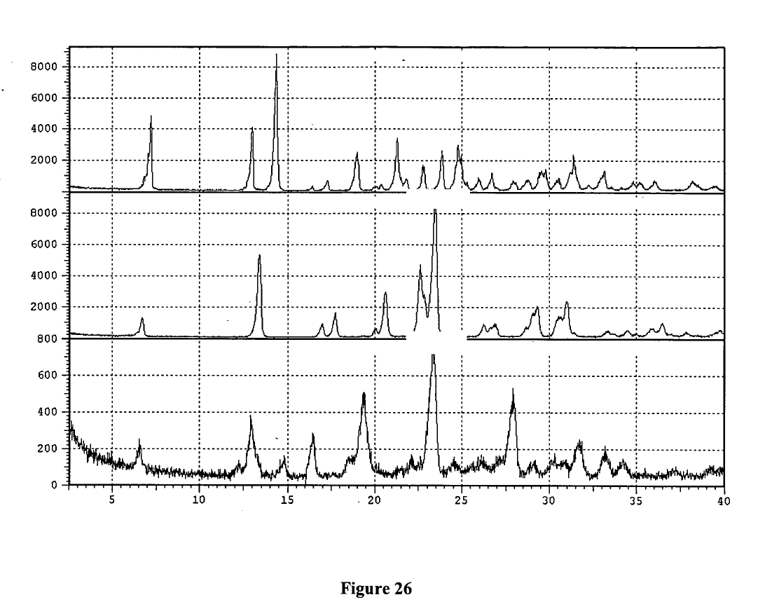

FIG. 26 illustrates a comparison XRPD pattern of decitabine polymorph forms A (top) B (middle) and C (bottom). The three polymorph forms of decitabine can be distinguished by the following distinguishing peaks. Form A has a sharp peak at °2θ value of roughly 7.0 whereas forms B and C have a minor peak in the same region. Form A has two peaks at °2θ values of roughly 13 and 14.5 as oppose to a single peak at °2θ value of roughly 13 in forms B and C. Form B has two peaks at °2θ values of roughly 22.5 and 26 as oppose to multiple short peaks in form A or one single peak at °2θ values of 26 in form C. And, form C has a sharp peak at °2θ value of 27 wherein forms A and B do not.

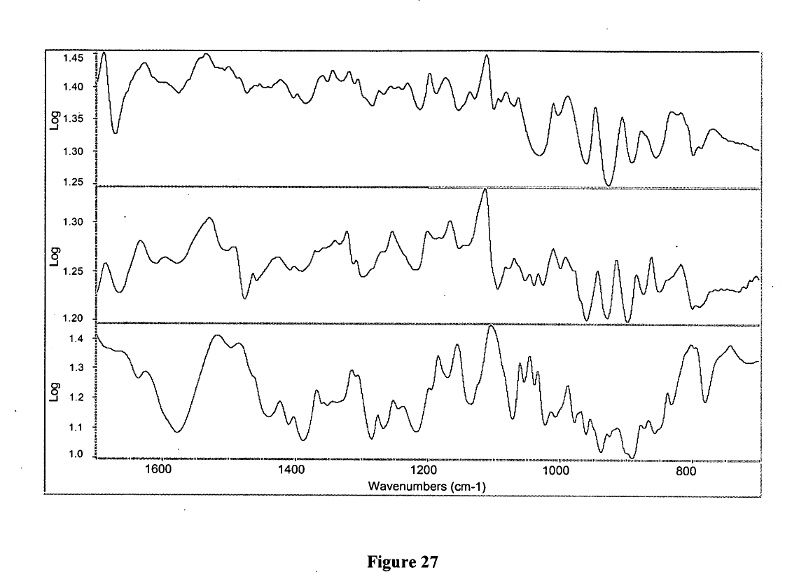

FIG. 27 illustrates a comparison of IR spectrum of decitabine forms A (top) B (middle) and C (bottom) between 1700 cm⁻¹ and 700 cm⁻¹. The IR spectra for each of the three polymorphs is unique and can be used to distinguish the polymorphs. For example, form A has a sharp peak at roughly 1700 cm⁻¹ which is a minor peak in form B and a broad peak in form C. Second, form B has a short peak at 1700 cm⁻¹, while both forms A and B have sharper peaks at that region. Third, form C has a broad peak between 1475 cm⁻¹ and 1550 cm⁻¹ and no peak at 1400 cm⁻¹ or 1600 cm⁻¹, while form A has a broad peak spanning the region of 1400 and 1600 and form B has a single peak at roughly 1550 cm⁻¹ and a shorter peak at 1450 cm⁻¹.

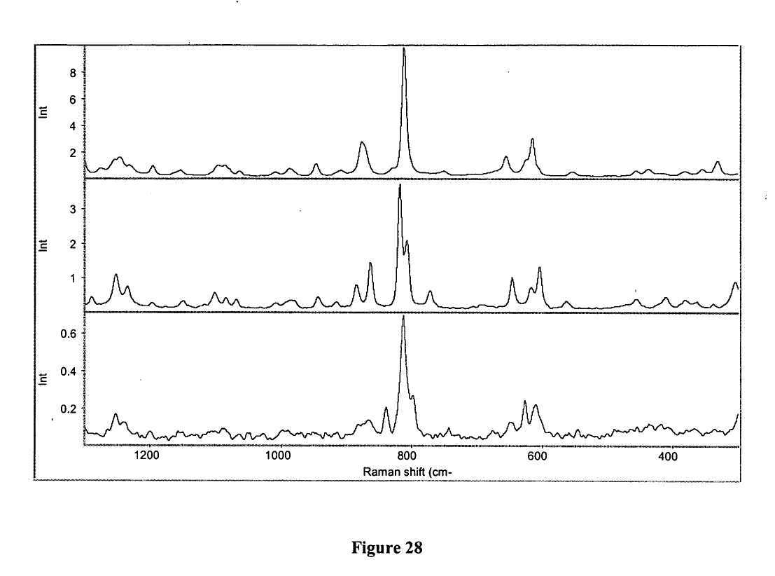

FIG. 28 illustrates a comparison of Raman spectrum of decitabine forms A (top) B (middle) and C (bottom). The spectra of each polymorph can be distinguished as follows. Form A has a sharp peak at roughly 800 cm⁻¹ while forms B and C have a split peak with a second shorter peak at roughly 800 cm⁻¹ and a sharper peak at a slightly lower shift (e.g., approximately 820 cm⁻¹). Second, polymorph form B has a short sharp peak at roughly 1300 while forms A and C have broader or shorter peaks in the same region. Furthermore, polymorph form C has no peaks between roughly 850 cm⁻¹ and 900 cm⁻¹, while both forms A and B have a short sharp peaks in that region.

What is claimed is:

1. A polymorph form of decitabine, the polymorph being characterizable as having an X-ray powder diffraction pattern with diffraction lines at °2θ values of approximately 7.0, 13, 14.5 for Cu Kα radiation of wavelength 1.5406 Angstrom.

2. The polymorphic form of decitabine according to claim 1 wherein the polymorphic form is further characterizable by differential scanning calorimetry, as having an endotherm at between 198.4° C. and 208.4° C. at a rate of 10° C./min.

3. The polymorphic form of decitabine according to claim 2 wherein the polymorphic form is further characterizable by differential scanning calorimetry, as having an endotherm at between 200.9° C. and 205.9° C. at a rate of 10° C./min.

4. The polymorphic form of decitabine according to claim 2 wherein the polymorphic form is further characterizable by differential scanning calorimetry, as having an endotherm at between 202.4° C. and 204.4° C. at a rate of 10° C./min.

5. The polymorph form of decitabine according to claim 1 wherein the polymorphic form comprises no more than a trace of water.

6. The polymorph form of decitabine according to claim 1 wherein the polymorphic form is anhydrous.

7. The polymorphic form of decitabine according to claim 1 wherein the polymorphic form is further characterizable as having an IR spectrum with minimal absorption between 3700 cm⁻¹ and 4000 cm⁻¹, a broad stretch between 3500 cm⁻¹ and 3000 cm⁻¹ with a peak at about 2000 cm⁻¹ and about 1850 cm⁻¹.

8. The polymorphic form of decitabine according to claim 1 wherein the polymorphic form is further characterizable as having a melt onset at approximately 198° C. and a melt at approximately 200° C.

9. The polymorphic form of decitabine according to claim 1 wherein the polymorphic form is further characterizable as being produced by cooling a solution of decitabine in methanol.

10. The polymorphic form of decitabine according to claim 1 wherein the polymorphic form is further characterizable as having a Raman spectra with a relatively weak stretch between about 2800 cm⁻¹ and 3000 cm⁻¹, a strong peak at around 800 cm⁻¹, encompassed by a series of small bands from about 600 cm⁻¹ to about 1600 cm⁻¹.

11. The polymorphic form of decitabine according to claim 1 further comprising a diffraction line at a °2θ value selected from the group consisting of approximately 18.5, 21.5 and 24.5 for Cu Kα radiation of wavelength 1.5406 Angstrom.

12. A polymorph form of decitabine being characterizable by having an X-ray powder diffraction pattern with diffraction lines at °2θ values of approximately 13.5, 22.5, and 23.5 for Cu Kα radiation of wavelength 1.5406 Angstrom.

13. The decitabine polymorph of claim 12 wherein said polymorph is further characterizable by having an X-ray powder diffraction line at °2θ value selected from the group consisting of approximately 6.5, 17, 18, and 20.5 for Cu Kα radiation of wavelength 1.5406 Angstrom.

14. The decitabine polymorph of claim 12 wherein said polymorph is further characterizable by differential scanning calorimetry as having an endotherm between 81.0° C. and 91.0° C., an endotherm between 89.9° C. and 99.9° C., and an endotherm between 193.4° C. and 203.4° C. at a rate of 10° C./min.

15. The decitabine polymorph of claim 12 wherein said polymorph is further characterizable by differential scanning calorimetry as having an endotherm between 83.5° C. and 88.5° C., an endotherm between 92.4° C. and 97.4° C., and an endotherm between 195.9° C. and 200.9° C. at a rate of 10° C./min.

16. The decitabine polymorph of claim 12 wherein said polymorph is further characterizable by differential scanning calorimetry as having an endotherm between 85.0° C. and 87.0° C., an endotherm between 93.9° C. and 95.9° C., and an endotherm between 197.4° C. and 199.4 C at a rate of 10° C./min.

17. The decitabine polymorph of claim 12 wherein said polymorph is further characterizable by having several additional week X-ray powder diffraction patterns with diffraction lines at °2θ values of approximately 18 and 20.5 for Cu Kα radiation of wavelength 1.5406 Angstrom.

18. The polymorph form of decitabine of claim 12 wherein the polymorph is further characterizable by having a weight loss of about 7.2% at 150° C.

19. The polymorph form of decitabine according to claim 12 wherein the polymorphic form is further characterizable by a structure as illustrated in FIG. 12.

20. The polymorph form of decitabine of claim 12 wherein the polymorph is further characterizable by having an IR spectrum with a broad stretch around 3400 cm⁻¹, a stretch between 3100 cm⁻¹ and 2800 cm⁻¹, a sharp peak at around 2000 cm⁻¹ and a complex fingerprint between about 1700 cm⁻¹ and 400 cm⁻¹.

21. The polymorph form of decitabine of claim 12 wherein the polymorph is further characterizable by a Raman spectra with a relatively weak stretch between about 3100 cm⁻¹ and 2900 cm⁻¹, a strong band around 800 cm⁻¹, and a series of small bands between 1600 cm⁻¹ and 600 cm⁻¹.

22. The polymorph form of decitabine of claim 12 wherein the polymorph is a monohydrate.

23. A polymorph form of decitabine being characterizable by having an X-ray powder diffraction pattern with diffraction lines at °2θ values of approximately 19, 23, and 27.5 for Cu Kα radiation of wavelength 1.5406 Angstrom.

24. The polymorph form of decitabine of claim 23 wherein the polymorph is further characterizable by having an X-ray powder diffraction pattern with a diffraction line at °2θ value selected from the group consisting of 13, 14.5, and 16.5.

25. The polymorph form of decitabine of claim 23 wherein the polymorph is further characterizable by differential scanning calorimetry as having an endotherm between 44.3° C. and 54.3° C., an endotherm between 159.6° C. and 169.6° C., and an endotherm between 190.8° C. and 200.8° C. at a rate of 10° C./min.

26. The polymorph form of decitabine of claim 23 wherein the polymorph is further characterizable by differential scanning calorimetry as having an endotherm between 46.8° C. and 52.8° C., an endotherm between 162.1° C. and 167.1° C., and an endotherm between 193.3° C. and 198.3° C. at a rate of 10° C./min.

27. The polymorph form of decitabine of claim 23 wherein the polymorph is further characterizable by differential scanning calorimetry as having an endotherm between 48.3° C. and 50.3° C., an endotherm between 163.6° C. and 165.6° C., and an endotherm between 194.8° C. and 196.8° C. at a rate of 10° C./min.

28. The polymorph form of decitabine of claim 23 wherein the polymorph is further characterizable by having an IR spectrum with minimal absorption between 3625 cm⁻¹, and 3675 cm⁻¹, a broad stretch at around 3400 cm⁻¹, a weak peak at around 2000 cm⁻¹ and a complex fingerprint between about 1700 cm⁻¹ and 500 cm⁻¹.

29. The polymorph form of decitabine of claim 23 wherein the polymorph is further characterizable by a Raman spectrum with a peak between about 3100 cm⁻¹ and 2800 cm⁻¹ and a peak at about 800 cm⁻¹.

30. The polymorph form of decitabine of claim 23 wherein the polymorph is prepared by vacuum evaporation of a solution of decitabine in 2,2,2-trifluoroethanol and water, followed by evaporation in ambient temperature and vacuum oven drying in ambient temperature.

31. A pharmaceutical composition comprising a pharmaceutical carrier and the decitabine polymorph of claims 1, 12 or 23.

32. A method for treating a patient having a neoplastic disease, the method comprising:
administering to a patient a pharmaceutically effective amount of the pharmaceutical composition of any one of claims 1, 12 or 23.

33. The method according to claim 32 wherein the neoplastic disease is selected from restenosis, benign tumor, cancer, hematological disorders, and atherosclerosis.

34. The method according to claim 33 wherein the benign tumor is selected from the group consisting of hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

35. The method according to claim 33 wherein the cancer is selected from the group consisting of breast cancer, skin cancer, bone cancer, prostate cancer, liver cancer, lung cancer, brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, and epidermoid carcinomas.

36. The method of claim 33 the hematological disorder is selected from the group consisting of acute myeloid leukemia, acute promyelocytic leukemia, acute lymphoblastic leukemia, chronic myelogenous leukemia, the myelodysplastic syndromes, and sickle cell anemia.

37. A method for crystallizing a decitabine polymorph comprising performing a crystallization process on decitabine wherein methanol is employed as the primary solvent to form crystalline of decitabine having an X-ray diffraction pattern of °2θ values of approximately 7.0, 13, and 14.5, for Cu Kα radiation of wavelength 1.5406 Angstrom.

38. The method of claim 37 wherein the polymorph is further characterizable by having an X-ray diffraction pattern of °2θ values of approximately 18.5, 21.5 and 24.5

39. A method for crystallizing a decitabine polymorph comprising performing a crystallization process on decitabine wherein methanol is employed as the primary solvent to form crystalline of decitabine having an X-ray diffraction pattern of °2θ values of approximately 13.5, 22.5, and 23.5 for Cu Kα radiation of wavelength 1.5406 Angstrom.

40. The method of claim 37 wherein the polymorph is further characterizable by having an X-ray powder diffraction line at °2θ value selected from the group consisting of approximately 6.5, 17, 18, and 20.5 for Cu Kα radiation of wavelength 1.5406 Angstrom.

41. A method for crystallizing a decitabine polymorph comprising performing a crystallization process on decitabine wherein methanol is employed as the primary solvent to form crystalline of decitabine having an X-ray diffraction pattern of °2θ values of approximately 19, 23, and 27.5, for Cu Kα radiation of wavelength 1.5406 Angstrom.

42. The method of claim 41 wherein the polymorph is further characterizable by having an X-ray powder diffraction line at °2θ value selected from the group consisting of approximately 13, 14.5, and 16.5 for Cu Kα radiation of wavelength 1.5406 Angstrom.
